(19) United States
(12) Patent Application Publication
(10) Pub. No.: US 2019/0099421 A1
(43) Pub. Date: Apr. 4, 2019

(54) CINNOLIN-4-AMINE COMPOUNDS AND THEIR USE IN TREATING CANCER

(71) Applicant: AstraZeneca AB, Södertälje (SE)

(72) Inventors: Kurt Gordon PIKE, Little Chesterford (GB); Bernard Christophe BARLAAM, Cambridge (GB)

(21) Appl. No.: 16/086,742

(22) PCT Filed: Mar. 20, 2017
(86) PCT No.: PCT/EP2017/056592

§ 371 Date: Sep. 20, 2018

Related U.S. Application Data
(60) Provisional application No. 62/310,883, filed on Mar. 21, 2016.

(51) Int. Cl. A61K 31/502; A61P 35/00

(52) U.S. Cl. CPC A61K 31/502; A61K 31/704; A61P 35/00

(57) ABSTRACT

This specification generally relates to compounds of Formula (I):

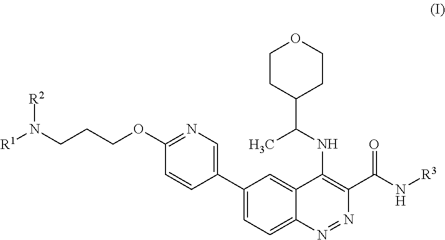

And pharmaceutically acceptable salts thereof, where R¹, R² and R³ have any of the meanings defined herein. The specification also relates to the use of such compounds and salts thereof to treat or prevent ATM kinase mediated disease, including cancer. The specification further relates to crystalline forms of compounds of Formula (I) and pharmaceutically acceptable salts thereof; pharmaceutical compositions comprising such compounds and salts thereof; kits comprising such compounds and salts thereof; methods of manufacture of such compounds and salts thereof; intermediates useful in the manufacture of such compounds and salts thereof; and to methods of treating ATM kinase mediated disease, including cancer, using compounds of Formula (I) and salts thereof alone or in combination with other therapies.

### FIELD OF INVENTION

This specification generally relates to substituted cinnolin-4-amine compounds and pharmaceutically acceptable salts thereof. These compounds selectively modulate ataxia telangiectasia mutated (“ATM”) kinase, and the specification therefore also relates to the use of such compounds and salts thereof to treat or prevent ATM kinase mediated disease, including cancer. The specification further relates to crystalline forms of substituted cinnolin-4-amine compounds and pharmaceutically acceptable salts thereof;

pharmaceutical compositions comprising such compounds and salts thereof; kits comprising such compounds and salts thereof; methods of manufacture of such compounds and salts thereof; intermediates useful in the manufacture of such compounds and salts thereof; and to methods of treating ATM kinase mediated disease, including cancer, using cinnolin-4-amine compounds and salts thereof alone or in combination with other therapies.

### BACKGROUND

ATM kinase is a serine threonine kinase originally identified as the product of the gene mutated in ataxia telangiectasia. Ataxia telangiectasia is located on human chromosome 11q22-23 and codes for a large protein of about 350 kDa, which is characterized by the presence of a phosphatidylinositol (“PI”) 3-kinase-like serine/threonine kinase domain flanked by FRAP-ATM-TRRAP and FATC domains which modulate ATM kinase activity and function. ATM kinase has been identified as a major player of the DNA damage response elicited by double strand breaks. It primarily functions in S/G2/M cell cycle transitions and at collapsed replication forks to initiate cell cycle checkpoints, chromatin modification, HR repair and pro-survival signalling cascades in order to maintain cell integrity after DNA damage (Lavin, 2008).

ATM kinase signalling can be broadly divided into two categories: a canonical pathway, which signals together with the Mre11-Rad50-NBS1 complex from double strand breaks and activates the DNA damage checkpoint, and several non-canonical modes of activation, which are activated by other forms of cellular stress (Cremona et al., 2013).

ATM kinase is rapidly and robustly activated in response to double strand breaks and is reportedly able to phosphorylate in excess of 800 substrates (Matsuoka et al., 2007), coordinating multiple stress response pathways (Kurz and Lees Miller, 2004). ATM kinase is present predominantly in the nucleus of the cell in an inactive homodimeric form but autophosphorylates itself on Ser1981 upon sensing a DNA double strand break (canonical pathway), leading to dissociation to a monomer with full kinase activity (Bakkenist et al., 2003). This is a critical activation event, and ATM phospho-Ser1981 is therefore both a direct pharmacodynamic and patient selection biomarker for tumour pathway dependency.

ATM kinase responds to direct double strand breaks caused by common anti-cancer treatments such as ionising radiation and topoisomerase-II inhibitors (for example doxorubicin or etoposide) but also to topoisomerase-I inhibitors (for example irinotecan or topotecan) via single strand break to double strand break conversion during replication. ATM kinase inhibition can potentiate the activity of any these agents, and as a result ATM kinase inhibitors are expected to be of use in the treatment of cancer.

### SUMMARY OF INVENTION

Briefly, this specification describes, in part, a compound of Formula (I):

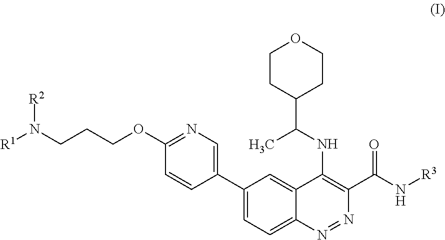

Or a pharmaceutically acceptable salt thereof, where:

R¹ is (C₁-C₃)alkyl;

R² is hydro or (C₁-C₃)alkyl; or

R¹ and R² together with the nitrogen atom to which they are attached form an azetidinyl, pyrrolidinyl, or piperidinyl ring; and

R³ is hydro or methyl.

This specification also describes, in part, a pharmaceutical composition which comprises a compound of Formula (I), or a pharmaceutically acceptable salt thereof, and at least one pharmaceutically acceptable diluent or carrier.

This specification also describes, in part, a compound of Formula (I), or a pharmaceutically acceptable salt thereof, for use in therapy.

This specification also describes, in part, a compound of Formula (I), or a pharmaceutically acceptable salt thereof, for use in the treatment of cancer.

This specification also describes, in part, a compound of Formula (I), or a pharmaceutically acceptable salt thereof, for the manufacture of a medicament for the treatment of cancer.

This specification also describes, in part, a method for treating cancer in a warm blooded animal in need of such treatment, which comprises administering to said warm-blooded animal a therapeutically effective amount of a compound of Formula (I), or a pharmaceutically acceptable salt thereof.

### BRIEF DESCRIPTION OF THE FIGURES

FIG. 1: X-Ray Powder Diffraction Pattern of Form A of 6-[6-(3-Dimethylaminopropoxy)pyridin-3-yl]-N-methyl-4-[[(1S)-1-(oxan-4-yl)ethyl]amino]cinnoline-3-carboxamide.

FIG. 2: DSC Thermogram of Form A of 6-[6-(3-Dimethylaminopropoxy)pyridin-3-yl]-N-methyl-4-[[(1S)-1-(oxan-4-yl)ethyl]amino]cinnoline-3-carboxamide.

FIG. 3: X-Ray Powder Diffraction Pattern of Form B of 6-[6-(3-Dimethylaminopropoxy)pyridin-3-yl]-N-methyl-4-[[(1S)-1-(oxan-4-yl)ethyl]amino]cinnoline-3-carboxamide.

FIG. 4: DSC Thermogram of Form B of 6-[6-(3-Dimethylaminopropoxy)pyridin-3-yl]-N-methyl-4-[[(1S)-1-(oxan-4-yl)ethyl]amino]cinnoline-3-carboxamide.

### DESCRIPTION OF ILLUSTRATIVE EMBODIMENTS

Many embodiments are detailed throughout the specification and will be apparent to a reader skilled in the art. The invention is not to be interpreted as being limited to any particular embodiment.

In the first embodiment there is provided a compound of Formula (I):

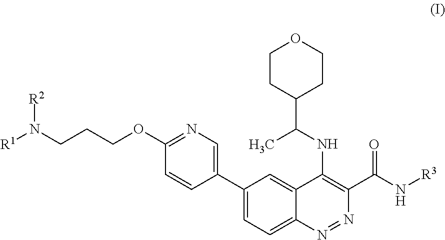

Or a pharmaceutically acceptable salt thereof, where:

R¹ is (C₁-C₃)alkyl;

R² is hydro or (C₁-C₃)alkyl; or

R¹ and R² together with the nitrogen atom to which they are attached form an azetidinyl, pyrrolidinyl, or piperidinyl ring; and

R³ is hydro or methyl.

Compounds and salts described in this specification may exist in optically active or racemic forms by virtue of their asymmetric carbon atom. The invention includes any optically active or racemic form of a compound of Formula (I) which possesses ATM kinase inhibitory activity, as for example measured using the tests described herein. The synthesis of optically active forms may be carried out by standard techniques of organic chemistry well known in the art, for example by synthesis using optically active materials or by resolution of a racemic form.

Therefore, in one embodiment there is provided a compound of Formula (IA):

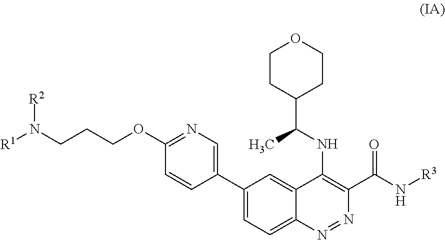

Or a pharmaceutically acceptable salt thereof, where:

R¹ is (C₁-C₃)alkyl;

R² is hydro or (C₁-C₃)alkyl; or

R¹ and R² together with the nitrogen atom to which they are attached form an azetidinyl, pyrrolidinyl, or piperidinyl ring; and

R³ is hydro or methyl.

In one embodiment there is provided a compound of Formula (IA), or a pharmaceutically acceptable salt thereof, which is in an enantiomeric excess (% ee) of ≥95%, ≥98% or ≥99%. In one embodiment there is provided a compound of Formula (IA), or a pharmaceutically acceptable salt thereof, which is in an enantiomeric excess (% ee) of ≥99%.

In one embodiment there is provided a compound of Formula (IB):

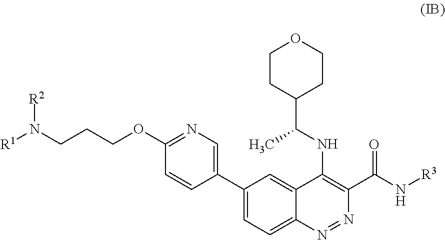

Or a pharmaceutically acceptable salt thereof, where:

R¹ is (C₁-C₃)alkyl;

R² is hydro or (C₁-C₃)alkyl; or

R¹ and R² together with the nitrogen atom to which they are attached form an azetidinyl, pyrrolidinyl, or piperidinyl ring; and

R³ is hydro or methyl.

In one embodiment there is provided a compound of Formula (IB), or a pharmaceutically acceptable salt thereof, which is in an enantiomeric excess (% ee) of ≥95%, ≥98% or ≥99%. In one embodiment there is provided a compound of Formula (IB), or a pharmaceutically acceptable salt thereof, which is in an enantiomeric excess (% ee) of ≥99%.

The term “(C₁-C₃)alkyl” refers to both straight-chain and branched-chain alkyl groups, and includes methyl, ethyl, propyl and isopropyl groups. However, any references to individual alkyl groups such as “propyl” are specific for the straight-chain version only, and references to individual branched-chain alkyl groups such as “isopropyl” are specific for the branched-chain version only.

Where it is mentioned that “R¹ and R² together with the nitrogen atom to which they are attached form an azetidinyl, pyrrolidinyl or piperidinyl ring”, this means the R¹ and R² groups are joined via a carbon-carbon covalent bond to form an unsubstituted alkylene chain of the appropriate length for the corresponding ring. For example, when R¹ and R² together with the nitrogen atom to which they are attached form a pyrrolidinyl ring, the R¹ and R² groups represent an unsubstituted butylene chain which is attached to the relevant nitrogen atom in Formula (I) (or Formula (IA) or Formula (IB) or in any other relevant embodiment) at both terminal carbons.

The term “pharmaceutically acceptable” is used to specify that an object (for example a salt, dosage form, diluent or carrier) is suitable for use in patients. An example list of pharmaceutically acceptable salts can be found in the Handbook of Pharmaceutical Salts: Properties, Selection and Use, P. H. Stahl and C. G. Wermuth, editors, Weinheim/Zürich: Wiley-VCH/VHCA, 2002. A suitable pharmaceutically acceptable salt of a compound of Formula (I), (IA) or (IB) is, for example, an acid-addition salt. An acid addition salt of a compound of Formula (I), (IA) or (IB) may be formed by bringing the compound into contact with a suitable inorganic or organic acid under conditions known to the skilled person. An acid addition salt may for example be formed using an inorganic acid selected from hydrochloric acid, hydrobromic acid, sulphuric acid and phosphoric acid. An acid addition salt may also for example be formed using an organic acid selected from trifluoroacetic acid, citric acid, maleic acid, oxalic acid, fumaric acid, tartaric acid, pyruvic acid, methanesulfonic acid, benzenesulfonic acid and para-toluenesulfonic acid. It is to be understood that it it may be possible to form salts with acids not specifically listed above, and that as a result the broadest definition of “pharmaceutically acceptable” is not to be limited to only salts formed with the specifically recited acids.

Therefore, in one embodiment there is provided a compound of Formula (I) or a pharmaceutically acceptable salt thereof, where the pharmaceutically acceptable salt is a hydrochloric acid, hydrobromic acid, sulphuric acid, phosphoric acid, trifluoroacetic acid, citric acid, maleic acid, oxalic acid, fumaric acid, tartaric acid, pyruvic acid, methanesulfonic acid, benzenesulfonic acid or para-toluenesulfonic acid salt. In one embodiment there is provided a compound of Formula (IA) or a pharmaceutically acceptable salt thereof, where the pharmaceutically acceptable salt is a hydrochloric acid, hydrobromic acid, sulphuric acid, phosphoric acid, trifluoroacetic acid, citric acid, maleic acid, oxalic acid, fumaric acid, tartaric acid, pyruvic acid, methanesulfonic acid, benzenesulfonic acid or para-toluenesulfonic acid salt. In one embodiment there is provided a compound of Formula (IB) or a pharmaceutically acceptable salt thereof, where the pharmaceutically acceptable salt is a hydrochloric acid, hydrobromic acid, sulphuric acid, phosphoric acid, trifluoroacetic acid, citric acid, maleic acid, oxalic acid, fumaric acid, tartaric acid, pyruvic acid, methanesulfonic acid, benzenesulfonic acid or para-toluenesulfonic acid salt.

A further embodiment provides any of the embodiments defined herein (for example the embodiment of claim 1) with the proviso that one or more specific Examples (for instance one, two or three specific Examples) selected from Examples 1, 2, 3, 4, 5 and 6 is individually disclaimed.

Some values of variable groups in Formulae (I), (IA) and (IB) are as follows. Such values may be used in combination with any of the definitions, claims (for example claim 1), or embodiments defined herein to provide further embodiments.
a) R¹ is (C₁-C₃)alkyl and R² is hydro or (C₁-C₃)alkyl; or R¹ and R² together with the nitrogen atom to which they are attached form a pyrrolidinyl ring.
b) R¹ is methyl and R² is hydro or methyl; or R¹ and R² together with the nitrogen atom to which they are attached form a pyrrolidinyl ring.
c) R¹ and R² are both methyl; or R¹ and R² together with the nitrogen atom to which they are attached form a pyrrolidinyl ring.
d) R¹ is methyl and R² is hydro or methyl.
e) R¹ and R² are both methyl.
f) R¹ and R² together with the nitrogen atom to which they are attached form a pyrrolidinyl ring.
g) R¹ is methyl.
h) R² is (C₁-C₃)alkyl.
i) R² is hydro or methyl.
j) R² is methyl.
k) R² is hydro.
l) R³ is hydro.
m) R³ is methyl.

In one embodiment of the invention there is provided a compound of Formula (I), or a pharmaceutically acceptable salt thereof, which is selected from:
6-[6-(3-Dimethylaminopropoxy)pyridin-3-yl]-N-methyl-4-[[(1S)-1-(oxan-4-yl)ethyl]amino]cinnoline-3-carboxamide;
6-[6-(3-Dimethylaminopropoxy)pyridin-3-yl]-4-[[(1S)-1-(oxan-4-yl)ethyl]amino]cinnoline-3-carboxamide;
4-[[(1S)-1-(Oxan-4-yl)ethyl]amino]-6-[6-(3-pyrrolidin-1-ylpropoxy)pyridin-3-yl]cinnoline-3-carboxamide;
6-[6-(3-Methylaminopropoxy)pyridin-3-yl]-4-[[(1S)-1-(oxan-4-yl)ethyl]amino]cinnoline-3-carboxamide;
N-Methyl-6-[6-(3-methylaminopropoxy)pyridin-3-yl]-4-[[(1S)-1-(oxan-4-yl)ethyl]amino]cinnoline-3-carboxamide; and
6-[6-(3-Dimethylaminopropoxy)pyridin-3-yl]-N-methyl-4-[[(1R)-1-(oxan-4-yl)ethyl]amino]cinnoline-3-carboxamide.

In one embodiment of the invention there is provided a compound of Formula (I), or a pharmaceutically acceptable salt thereof, which is selected from:
6-[6-(3-Dimethylaminopropoxy)pyridin-3-yl]-N-methyl-4-[[(1S)-1-(oxan-4-yl)ethyl]amino]cinnoline-3-carboxamide;
6-[6-(3-Dimethylaminopropoxy)pyridin-3-yl]-4-[[(1S)-1-(oxan-4-yl)ethyl]amino]cinnoline-3-carboxamide;
4-[[(1S)-1-(Oxan-4-yl)ethyl]amino]-6-[6-(3-pyrrolidin-1-ylpropoxy)pyridin-3-yl]cinnoline-3-carboxamide;
6-[6-(3-Methylaminopropoxy)pyridin-3-yl]-4-[[(1S)-1-(oxan-4-yl)ethyl]amino]cinnoline-3-carboxamide; and
N-Methyl-6-[6-(3-methylaminopropoxy)pyridin-3-yl]-4-[[(1S)-1-(oxan-4-yl)ethyl]amino]cinnoline-3-carboxamide.

In one embodiment there is provided a compound of Formula (I), or a pharmaceutically acceptable salt thereof, where the compound is 6-[6-(3-Dimethylaminopropoxy)pyridin-3-yl]-N-methyl-4-[[(1R)-1-(oxan-4-yl)ethyl]amino]cinnoline-3-carboxamide.

In one embodiment of the invention there is provided 6-[6-(3-dimethylaminopropoxy)pyridin-3-yl]-N-methyl-4-[[(1S)-1-(oxan-4-yl)ethyl]amino]cinnoline-3-carboxamide, or a pharmaceutically acceptable salt thereof.

In one embodiment of the invention there is provided 6-[6-(3-dimethylaminopropoxy)pyridin-3-yl]-N-methyl-4-[[(1S)-1-(oxan-4-yl)ethyl]amino]cinnoline-3-carboxamide.

In one embodiment of the invention there is provided a pharmaceutically acceptable salt of 6-[6-(3-dimethylaminopropoxy)pyridin-3-yl]-N-methyl-4-[[(1S)-1-(oxan-4-yl)ethyl]amino]cinnoline-3-carboxamide.

In one embodiment of the invention there is provided 4-[[(1S)-1-(oxan-4-yl)ethyl]amino]-6-[6-(3-pyrrolidin-1-ylpropoxy)pyridin-3-yl]cinnoline-3-carboxamide, or a pharmaceutically acceptable salt thereof.

In one embodiment of the invention there is provided 4-[[(1S)-1-(oxan-4-yl)ethyl]amino]-6-[6-(3-pyrrolidin-1-ylpropoxy)pyridin-3-yl]cinnoline-3-carboxamide.

In one embodiment of the invention there is provided a pharmaceutically acceptable salt of 4-[[(1S)-1-(oxan-4-yl)ethyl]amino]-6-[6-(3-pyrrolidin-1-ylpropoxy)pyridin-3-yl]cinnoline-3-carboxamide.

Compounds and salts described in this specification may exist in solvated forms and unsolvated forms. For example, a solvated form may be a hydrated form, such as a hemi-hydrate, a mono-hydrate, a di-hydrate, a tri-hydrate or an alternative quantity thereof. The invention encompasses all such solvated and unsolvated forms of compounds of Formula (I), (IA), or (IB), particularly to the extent that such forms possess ATM kinase inhibitory activity, as for example measured using the tests described herein.

Atoms of the compounds and salts described in this specification may exist as their isotopes. The invention encompasses all compounds of Formula (I), (IA), or (IB) where an atom is replaced by one or more of its isotopes (for example a compound of Formula (I), (IA), or (IB) where one or more carbon atom is an ¹¹C or ¹³C carbon isotope, or where one or more hydrogen atoms is a ²H or ³H isotope).

Compounds and salts described in this specification may exist as a mixture of tautomers. “Tautomers” are structural isomers that exist in equilibrium resulting from the migration of a hydrogen atom. The invention includes all tautomers of compounds of Formula (I), (IA), or (IB) particularly to the extent that such tautomers possess ATM kinase inhibitory activity.

Compounds and salts described in this specification may be crystalline, and may exhibit one or more crystalline forms. The invention encompasses any crystalline or amorphous form of a compound of Formula (I), (IA), or (IB), or mixture of such forms, which possesses ATM kinase inhibitory activity.

It is generally known that crystalline materials may be characterised using conventional techniques such as X-Ray Powder Diffraction (XRPD), Differential Scanning calorimetry (DSC), Thermal Gravimetric Analysis (TGA), Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

The specific crystalline forms described herein provide XRPD patterns substantially the same as the XRPD patterns shown in the Figures, and have the various 2-theta values as shown in the Tables included herein. One skilled in the art will understand that an XRPD pattern or diffractogram may be obtained which has one or more measurement errors depending on the recording conditions, such as the equipment or machine used. Similarly, it is generally known that intensities in an XRPD pattern may fluctuate depending on measurement conditions or sample preparation as a result of preferred orientation. Persons skilled in the art of XRPD will further realise that the relative intensity of peaks can also be affected by, for example, grains above 30 μm in size and non-unitary aspect ratios. The skilled person understands that the position of reflections can be affected by the precise height at which the sample sits in the diffractometer, and also the zero calibration of the diffractometer. The surface planarity of the sample may also have a small effect.

As a result of these considerations, the diffraction pattern data presented are not to be taken as absolute values (Jenkins, R & Snyder, R. L. ‘Introduction to X-Ray Powder Diffractometry’ John Wiley & Sons 1996; Bunn, C. W. (1948), ‘Chemical Crystallography’, Clarendon Press, London; Klug, H. P. & Alexander, L. E. (1974), ‘X-Ray Diffraction Procedures’). It should correspondingly be understood that the crystalline forms embodied herein are not limited to those that provide XRPD patterns that are identical to the XRPD pattern shown in the Figures, and any crystals providing XRPD patterns substantially the same as those shown in the Figures fall within the scope of the corresponding embodiment. A person skilled in the art of XRPD is able to judge the substantial identity of XRPD patterns. Generally, a measurement error of a diffraction angle in an XRPD is approximately plus or minus 0.2° 2-theta, and such degree of a measurement error should be taken into account when considering the X-ray powder diffraction pattern in the Figures and when reading data contained in the Tables included herein.

A person skilled in the art also understands that the value or range of values observed in a particular compound's DSC Thermogram will show variation between batches of different purities. Therefore, whilst for one compound the range may be small, for others the range may be quite large. Generally, a measurement error of a diffraction angle in DSC thermal events is approximately plus or minus 5° C., and such degree of a measurement error should be taken into account when considering the DSC data included herein. TGA thermograms show similar variations, such that a person skilled in the art recognises that measurement errors should be taken into account when judging substantial identity of TGA thermograms.

The compound of Example 1 exhibits crystalline properties, and two crystalline forms are characterised herein.

In one embodiment there is provided a crystalline form, Form A, of 6-[6-(3-dimethylaminopropoxy)pyridin-3-yl]-N-methyl-4-[[(1S)-1-(oxan-4-yl)ethyl]amino]cinnoline-3-carboxamide.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least one specific peak at about 2-theta=4.9°.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least one specific peak at about 2-theta=8.1°.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least two specific peaks at about 2-theta=4.9 and 8.1°.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising specific peaks at about 2-theta=4.9, 8.1, 9.8, 10.6, 14.5, 15.6, 18.8, 20.8, 21.3 and 23.8°.

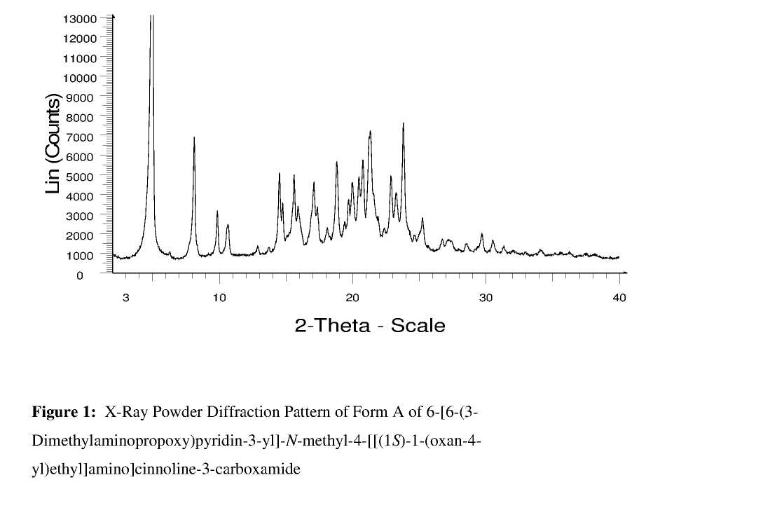

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern substantially the same as the X-ray powder diffraction pattern shown in FIG. 1.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least one specific peak at 2-theta=4.9° plus or minus 0.2° 2-theta.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least one specific peak at 2-theta=8.1° plus or minus 0.2° 2-theta.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least two specific peaks at 2-theta=4.9 and 8.1° plus or minus 0.2° 2-theta.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising specific peaks at 2-theta=4.9, 8.1, 9.8, 10.6, 14.5, 15.6, 18.8, 20.8, 21.3 and 23.8° plus or minus 0.2° 2-theta.

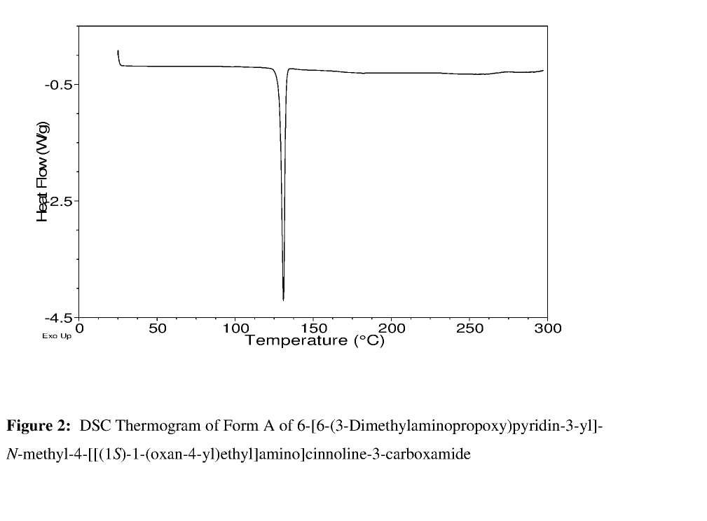

DSC analysis of Form A of 6-[6-(3-dimethylaminopropoxy)pyridin-3-yl]-N-methyl-4-[[(1S)-1-(oxan-4-yl)ethyl]amino]cinnoline-3-carboxamide shows a melting endotherm with an onset of about 128.7° C. and a peak at about 131.0° C. (FIG. 2).

Therefore, in one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has a DSC thermogram comprising an endotherm with an onset of melting at about 128.7° C. and a peak at about 131.0° C.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has a DSC thermogram comprising an endotherm with an onset of melting at 128.7° C. plus or minus 5° C. and a peak at 131.0° C. plus or minus 5° C.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has a DSC thermogram comprising an endotherm with an onset of melting at 128.7° C. and a peak at 131.0° C.

In one embodiment there is provided a crystalline form, Form A of 6-[6-(3-dimethylaminopropoxy)pyridin-3-yl]-N-methyl-4-[[(1S)-1-(oxan-4-yl)ethyl]amino]cinnoline-3-carboxamide, which has a DSC thermogram substantially as shown in FIG. 2.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least one specific peak at about 2-theta=5.4°.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least one specific peak at about 2-theta=17.6°.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least two specific peaks at about 2-theta=5.4 and 17.6°.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising specific peaks at about 2-theta=5.4, 8.9, 9.5, 12.6, 17.0, 17.6, 21.6, 21.9, 23.2 and 23.4°.

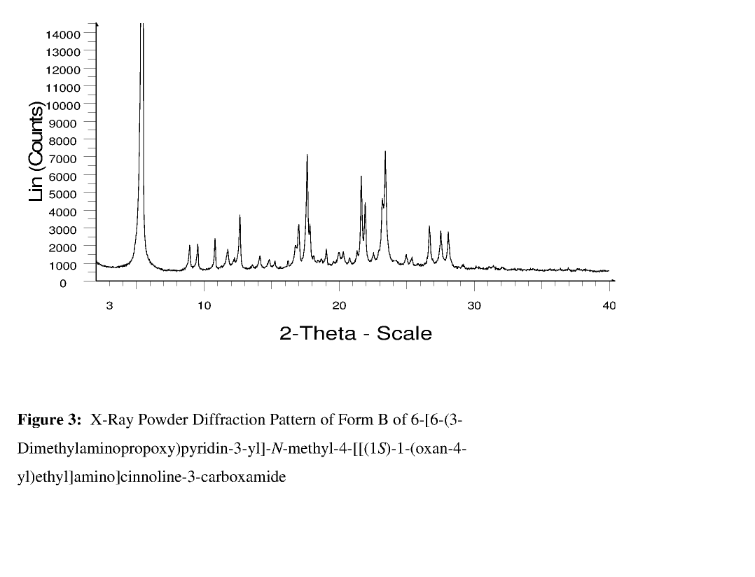

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern substantially the same as the X-ray powder diffraction pattern shown in FIG. 3.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least one specific peak at 2-theta=5.4° plus or minus 0.2° 2-theta.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least one specific peak at 2-theta=17.6° plus or minus 0.2° 2-theta.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising at least two specific peaks at 2-theta=5.4 and 17.6° plus or minus 0.2° 2-theta. In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has an X-ray powder diffraction pattern comprising specific peaks at 2-theta=5.4, 8.9, 9.5, 12.6, 17.0, 17.6, 21.6, 21.9, 23.2 and 23.4° plus or minus 0.2° 2-theta.

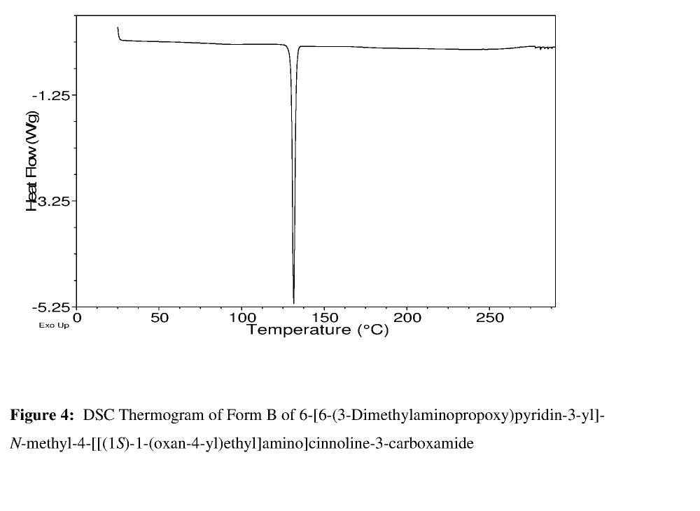

DSC analysis of Form B of 6-[6-(3-dimethylaminopropoxy)pyridin-3-yl]-N-methyl-4-[[(1S)-1-(oxan-4-yl)ethyl]amino]cinnoline-3-carboxamide shows a melting endotherm with an onset of about 130.0° C. and a peak at about 131.5° C. (FIG. 4).

Therefore, in one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has a DSC thermogram comprising an endotherm with an onset of melting at about 130.0° C. and a peak at about 131.5° C.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has a DSC thermogram comprising an endotherm with an onset of melting at 130.0° C. plus or minus 5° C. and a peak at 131.5° C. plus or minus 5° C.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has a DSC thermogram comprising an endotherm with an onset of melting at 130.0° C. and a peak at 131.5° C.

In one embodiment there is provided a crystalline form, Form B of 6-[6-(3-dimethylaminopropoxy)pyridin-3-yl]-N-methyl-4-[[(1S)-1-(oxan-4-yl)ethyl]amino]cinnoline-3-carboxamide, which has a DSC thermogram substantially as shown in FIG. 4.

When it is stated that an embodiment relates to a crystalline form, the degree of crystallinity may vary. Therefore, in one embodiment there is provided a crystalline form where the degree of crystallinity is greater than about 60%. In one embodiment the degree of crystallinity is greater than about 80%. In one embodiment the degree of crystallinity is greater than about 90%. In one embodiment the degree of crystallinity is greater than about 95%. In one embodiment the degree of crystallinity is greater than about 98%.

Compounds of Formula (I) may for example be prepared by the reaction of a compound of Formula (II):

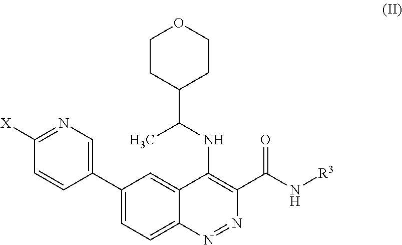

Or a salt thereof, where R³ is as defined in any of the embodiments herein and X is a leaving group (for example a halogen atom, or alternatively a fluorine atom) with a compound of formula (III):

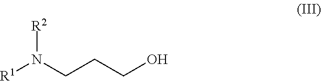

Or a salt thereof, where R¹ and R² are as defined in any of the embodiments herein. The reaction is conveniently performed in a suitable solvent (for example DMF, DMA or THF) and in the presence of a base (for example sodium hydride) at a suitable temperature (for example a temperature in the range of about 20-50° C.).

Compounds of Formula (II) are therefore useful as intermediates in the preparation of the compounds of Formula (I) and provide a further embodiment.

In one embodiment there is provided a compound of Formula (II), or a salt thereof, where:

R³ is hydro or methyl; and

X is a leaving group. In one embodiment X is a halogen atom or a triflate group. In one embodiment X is a fluorine atom.

Compounds of Formula (IA) may for example be prepared by the reaction of a compound of Formula (IIA):

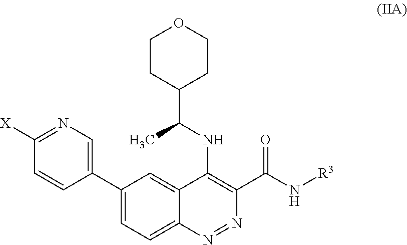

Or a salt thereof, where R³ is as defined in any of the embodiments herein and X is a leaving group (for example a halogen atom, or alternatively a fluorine atom) with a compound of formula (III):

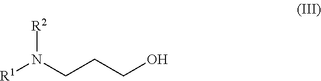

Or a salt thereof, where R¹ and R² are as defined in any of the embodiments herein. The reaction is conveniently performed in a suitable solvent (for example DMF, DMA or THF) and in the presence of a base (for example sodium hydride) at a suitable temperature (for example a temperature in the range of about 20-50° C.).

Compounds of Formula (IIA) are therefore useful as intermediates in the preparation of the compounds of Formula (IA) and provide a further embodiment.

In one embodiment there is provided a compound of Formula (IIA), or a salt thereof, where:

R³ is hydro or methyl; and

X is a leaving group. In one embodiment X is a halogen atom or a triflate group. In one embodiment X is a fluorine atom.

In one embodiment there is provided 6-(6-fluoropyridin-3-yl)-N-methyl-4-[[(1S)-1-(oxan-4-yl)ethyl]amino]cinnoline-3-carboxamide.

In one embodiment there is provided 6-(6-fluoropyridin-3-yl)-4-[[(1S)-1-(oxan-4-yl)ethyl]amino]cinnoline-3-carboxamide.

Compounds of Formula (IB) may for example be prepared by the reaction of a compound of Formula (IIB):

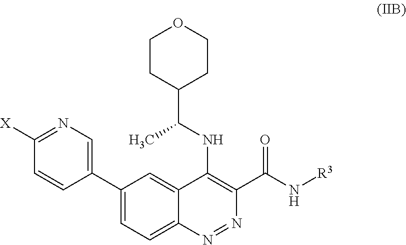

Or a salt thereof, where R³ is as defined in any of the embodiments herein and X is a leaving group (for example a halogen atom, or alternatively a fluorine atom) with a compound of formula (III):

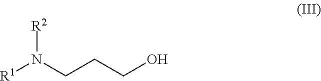

Or a salt thereof, where R¹ and R² are as defined in any of the embodiments herein. The reaction is conveniently performed in a suitable solvent (for example DMF, DMA or THF) and in the presence of a base (for example sodium hydride) at a suitable temperature (for example a temperature in the range of about 20-50° C.).

Compounds of Formula (IIB) are therefore useful as intermediates in the preparation of the compounds of Formula (IB) and provide a further embodiment.

In one embodiment there is provided a compound of Formula (IIB), or a salt thereof, where:

R³ is hydro or methyl; and

X is a leaving group. In one embodiment X is a halogen atom or a triflate group. In one embodiment X is a fluorine atom.

Compounds of Formula (I) may also be prepared by the reaction of a compound of Formula (IV):

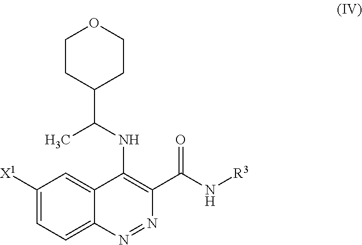

Or a salt thereof, where R³ is as defined in any of the embodiments herein and X¹ is an iodine, bromine, or chlorine atom or a triflate group, or alternatively a bromine atom, with a compound of formula (V):

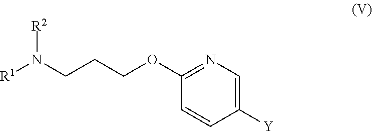

Or a salt thereof, where R¹ and R² are as defined in any of the embodiments herein and Y is a boronic acid, boronic ester or potassium trifluoroborate group (for example boronic acid, boronic acid pinacol ester, or potassium trifluoroborate). The reaction may be performed under standard conditions well known to those skilled in the art, for example in the presence of a palladium source (for example tetrakis triphenylphosphine palladium or palladium(II) acetate), optionally a phosphine ligand (for example Xantphos or S-phos), and a suitable base (for example cesium carbonate or triethylamine).

Compounds of Formula (IV) are therefore useful as intermediates in the preparation of the compounds of Formula (I) and provide a further embodiment.

In one embodiment there is provided a compound of Formula (IV), or a salt thereof, where:

R³ is hydro or methyl; and

X¹ is an iodine, bromine, or chlorine atom or a triflate group. In one embodiment X¹ is a bromine atom.

Compounds of Formula (IA) may also be prepared by the reaction of a compound of Formula (IVA):

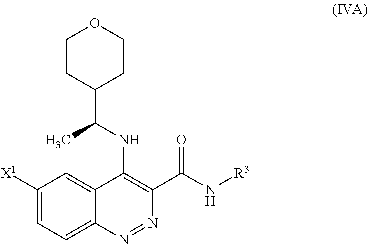

Or a salt thereof, where R³ is as defined in any of the embodiments herein and X¹ is an iodine, bromine, or chlorine atom or a triflate group, or alternatively a bromine atom, with a compound of formula (V):

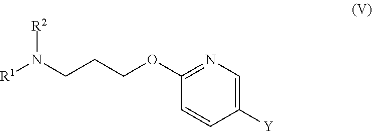

Or a salt thereof, where R¹ and R² are as defined in any of the embodiments herein and Y is a boronic acid, boronic ester or potassium trifluoroborate group (for example boronic acid, boronic acid pinacol ester, or potassium trifluoroborate). The reaction may be performed under standard conditions well known to those skilled in the art, for example in the presence of a palladium source (for example tetrakis triphenylphosphine palladium or palladium(II) acetate), optionally a phosphine ligand (for example Xantphos or S-phos), and a suitable base (for example cesium carbonate or triethylamine).

Compounds of Formula (IVA) are therefore useful as intermediates in the preparation of the compounds of Formula (I) and provide a further embodiment.

In one embodiment there is provided a compound of Formula (IVA), or a salt thereof, where:

R³ is hydro or methyl; and

X¹ is an iodine, bromine, or chlorine atom or a triflate group. In one embodiment X¹ is a bromine atom.

In one embodiment there is provided 6-bromo-N-methyl-4-[[(1S)-1-(oxan-4-yl)ethyl]amino]cinnoline-3-carboxamide.

In one embodiment there is provided 6-bromo-4-[[(1S)-1-(oxan-4-yl)ethyl]amino]cinnoline-3-carboxamide.

Compounds of Formula (IB) may also be prepared by the reaction of a compound of Formula (IVB):

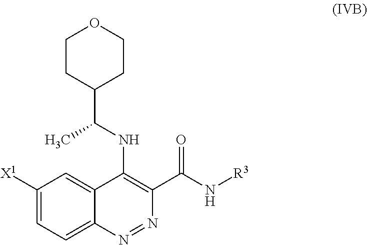

Or a salt thereof, where R³ is as defined in any of the embodiments herein and X¹ is an iodine, bromine, or chlorine atom or a triflate group, or alternatively a bromine atom, with a compound of formula (V):

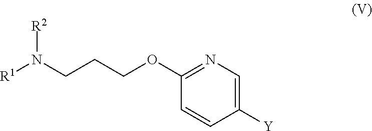

Or a salt thereof, where R¹ and R² are as defined in any of the embodiments herein and Y is a boronic acid, boronic ester or potassium trifluoroborate group (for example boronic acid, boronic acid pinacol ester, or potassium trifluoroborate). The reaction may be performed under standard conditions well known to those skilled in the art, for example in the presence of a palladium source (for example tetrakis triphenylphosphine palladium or palladium(II) acetate), optionally a phosphine ligand (for example Xantphos or S-phos), and a suitable base (for example cesium carbonate or triethylamine).

Compounds of Formula (IVB) are therefore useful as intermediates in the preparation of the compounds of Formula (IB) and provide a further embodiment.

In one embodiment there is provided a compound of Formula (IVB), or a salt thereof, where:

R³ is hydro or methyl; and

X¹ is an iodine, bromine, or chlorine atom or a triflate group. In one embodiment X¹ is a bromine atom.

In any of the embodiments where a compound of Formula (II), (IIA), (IIB), (IV), (IVA) or (IVB) or a salt of each of these compounds is mentioned it is to be understood that such salts do not need to be pharmaceutically acceptable salts. A suitable salt of a compound of Formula (II), (IIA), (IIB), (IV), (IVA) or (IVB) is, for example, an acid-addition salt. An acid addition salt of a compound of compound of Formula (II), (IIA), (IIB), (IV), (IVA) or (IVB) may be formed by bringing the compound into contact with a suitable inorganic or organic acid under conditions known to the skilled person. An acid addition salt may for example be formed using an inorganic acid selected from hydrochloric acid, hydrobromic acid, sulphuric acid and phosphoric acid. An acid addition salt may also be formed using an organic acid selected from trifluoroacetic acid, citric acid, maleic acid, oxalic acid, fumaric acid, tartaric acid, pyruvic acid, methanesulfonic acid, benzenesulfonic acid and para-toluenesulfonic acid.

Therefore, in one embodiment there is provided a compound of Formula (II) or a salt thereof, where the salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid or para-toluenesulfonic acid salt. In one embodiment there is provided a compound of Formula (IIA) or a salt thereof, where the salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid or para-toluenesulfonic acid salt. In one embodiment there is provided a compound of Formula (IIB) or a salt thereof, where the salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid or para-toluenesulfonic acid salt.

Therefore, in one embodiment there is provided a compound of Formula (IV) or a salt thereof, where the salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid or para-toluenesulfonic acid salt. In one embodiment there is provided a compound of Formula (IVA) or a salt thereof, where the salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid or para-toluenesulfonic acid salt. In one embodiment there is provided a compound of Formula (IVB) or a salt thereof, where the salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid or para-toluenesulfonic acid salt.

As a result of their ATM kinase inhibitory activity, the compounds of Formula (I), (IA), or (IB), and pharmaceutically acceptable salts thereof are expected to be useful in therapy, for example in the treatment of diseases or medical conditions mediated at least in part by ATM kinase, including cancer.

Where “cancer” is mentioned, this includes both non-metastatic cancer and also metastatic cancer, such that treating cancer involves treatment of both primary tumours and also tumour metastases.

“ATM kinase inhibitory activity” refers to a decrease in the activity of ATM kinase as a direct or indirect response to the presence of a compound of Formula (I), or pharmaceutically acceptable salt thereof, relative to the activity of ATM kinase in the absence of compound of Formula (I), (IA), or (IB), and pharmaceutically acceptable salts thereof. Such a decrease in activity may be due to the direct interaction of the compound of Formula (I), (IA), or (IB), and pharmaceutically acceptable salts thereof with ATM kinase, or due to the interaction of the compound of Formula (I), (IA), or (IB), and pharmaceutically acceptable salts thereof with one or more other factors that in turn affect ATM kinase activity. For example, the compound of Formula (I), (IA), or (IB), and pharmaceutically acceptable salts thereof may decrease ATM kinase by directly binding to the ATM kinase, by causing (directly or indirectly) another factor to decrease ATM kinase activity, or by (directly or indirectly) decreasing the amount of ATM kinase present in the cell or organism.

The term “therapy” is intended to have its normal meaning of dealing with a disease in order to entirely or partially relieve one, some or all of its symptoms, or to correct or compensate for the underlying pathology. The term “therapy” also includes “prophylaxis” unless there are specific indications to the contrary. The terms “therapeutic” and “therapeutically” should be interpreted in a corresponding manner.

The term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

In one embodiment there is provided a compound of Formula (I), or a pharmaceutically acceptable salt thereof, for use in therapy. In one embodiment there is provided a compound of Formula (IA), or a pharmaceutically acceptable salt thereof, for use in therapy. In one embodiment there is provided a compound of Formula (IB), or a pharmaceutically acceptable salt thereof, for use in therapy.

In one embodiment there is provided the use of the compound of Formula (I), or a pharmaceutically acceptable salt thereof, for the manufacture of a medicament. In one embodiment there is provided the use of the compound of Formula (IA), or a pharmaceutically acceptable salt thereof, for the manufacture of a medicament. In one embodiment there is provided the use of the compound of Formula (IB), or a pharmaceutically acceptable salt thereof, for the manufacture of a medicament.

In one embodiment there is provided a compound of Formula (I), or a pharmaceutically acceptable salt thereof, for use in the treatment of a disease mediated by ATM kinase. In one embodiment there is provided a compound of Formula (IA), or a pharmaceutically acceptable salt thereof, for use in the treatment of a disease mediated by ATM kinase. In one embodiment there is provided a compound of Formula (IB), or a pharmaceutically acceptable salt thereof, for use in the treatment of a disease mediated by ATM kinase. In any embodiment, said disease mediated by ATM kinase is cancer. In any embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In any embodiment, the cancer is colorectal cancer.

In one embodiment there is provided a compound of Formula (I), or a pharmaceutically acceptable salt thereof, for use in the treatment of cancer. In one embodiment there is provided a compound of Formula (IA), or a pharmaceutically acceptable salt thereof, for use in the treatment of cancer. In one embodiment there is provided a compound of Formula (IB), or a pharmaceutically acceptable salt thereof, for use in the treatment of cancer.

In one embodiment there is provided the use of the compound of Formula (I), or a pharmaceutically acceptable salt thereof, for the manufacture of a medicament for the treatment of a disease mediated by ATM kinase. In one embodiment there is provided the use of the compound of Formula (IA), or a pharmaceutically acceptable salt thereof, for the manufacture of a medicament for the treatment of a disease mediated by ATM kinase. In one embodiment there is provided the use of the compound of Formula (IB), or a pharmaceutically acceptable salt thereof, for the manufacture of a medicament for the treatment of a disease mediated by ATM kinase. In any embodiment, said disease mediated by ATM kinase is cancer. In any embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In any embodiment, the cancer is colorectal cancer.

In one embodiment there is provided the use of the compound of Formula (I), or a pharmaceutically acceptable salt thereof, for the manufacture of a medicament for the treatment of cancer. In one embodiment there is provided the use of the compound of Formula (IA), or a pharmaceutically acceptable salt thereof, for the manufacture of a medicament for the treatment of cancer. In one embodiment there is provided the use of the compound of Formula (IB), or a pharmaceutically acceptable salt thereof, for the manufacture of a medicament for the treatment of cancer.

In one embodiment there is provided a method for treating a disease in which inhibition of ATM kinase is beneficial in a warm-blooded animal in need of such treatment, which comprises administering to said warm-blooded animal a therapeutically effective amount of a compound of Formula (I), or a pharmaceutically acceptable salt thereof. In one embodiment there is provided a method for treating a disease in which inhibition of ATM kinase is beneficial in a warm-blooded animal in need of such treatment, which comprises administering to said warm-blooded animal a therapeutically effective amount of a compound of Formula (IA), or a pharmaceutically acceptable salt thereof. In one embodiment there is provided a method for treating a disease in which inhibition of ATM kinase is beneficial in a warm-blooded animal in need of such treatment, which comprises administering to said warm-blooded animal a therapeutically effective amount of a compound of Formula (IB), or a pharmaceutically acceptable salt thereof. In any embodiment, said disease mediated by ATM kinase is cancer. In any embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In any embodiment, the cancer is colorectal cancer.

The term “therapeutically effective amount” refers to an amount of a compound of Formula (I), (IA), or (IB), or corresponding pharmaceutically acceptable salts thereof which is effective to provide “therapy” in a subject, or to “treat” a disease or disorder in a subject. In the case of cancer, the therapeutically effective amount may cause any of the changes observable or measurable in a subject as described in the definition of “therapy”, “treatment” and “prophylaxis” above. For example, the effective amount can reduce the number of cancer or tumour cells; reduce the overall tumour size; inhibit or stop tumour cell infiltration into peripheral organs including, for example, the soft tissue and bone; inhibit and stop tumour metastasis; inhibit and stop tumour growth; relieve to some extent one or more of the symptoms associated with cancer; reduce morbidity and mortality; improve quality of life; or a combination of such effects. An effective amount may be an amount sufficient to decrease the symptoms of a disease responsive to inhibition of ATM kinase activity. For cancer therapy, efficacy in-vivo can, for example, be measured by assessing the duration of survival, time to disease progression (TTP), the response rates (RR), duration of response, and/or quality of life. As recognized by those skilled in the art, effective amounts may vary depending on route of administration, excipient usage, and co-usage with other agents. For example, where a combination therapy is used, the amount of the compound of Formula (I), (IA), or (IB), or corresponding pharmaceutically acceptable salts thereof and the amount of the other pharmaceutically active agent(s) are, when combined, jointly effective to treat a targeted disorder in the animal patient. In this context, the combined amounts are in a “therapeutically effective amount” if they are, when combined, sufficient to decrease the symptoms of a disease responsive to inhibition of ATM activity as described above. Typically, such amounts may be determined by one skilled in the art by, for example, starting with the dosage range described in this specification for the compound of Formula (I), (IA), or (IB), or corresponding pharmaceutically acceptable salts thereof and an approved or otherwise published dosage range(s) of the other pharmaceutically active compound(s).

“Warm-blooded animals” include, for example, humans.

In one embodiment there is provided a method for treating cancer in a warm-blooded animal in need of such treatment, which comprises administering to said warm-blooded animal a therapeutically effective amount of a compound of Formula (I), or a pharmaceutically acceptable salt thereof. In one embodiment there is provided a method for treating cancer in a warm-blooded animal in need of such treatment, which comprises administering to said warm-blooded animal a therapeutically effective amount of a compound of Formula (IA), or a pharmaceutically acceptable salt thereof. In one embodiment there is provided a method for treating cancer in a warm-blooded animal in need of such treatment, which comprises administering to said warm-blooded animal a therapeutically effective amount of a compound of Formula (IB), or a pharmaceutically acceptable salt thereof. In any embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In any embodiment, the cancer is colorectal cancer.

The anti-cancer treatment described in this specification may be useful as a sole therapy, or may involve, in addition to administration of the compound of Formula (I), (IA), or (IB), or corresponding pharmaceutically acceptable salts thereof conventional surgery, radiotherapy or chemotherapy; or a combination of such additional therapies. Such conventional surgery, radiotherapy or chemotherapy may be used simultaneously, sequentially or separately to treatment with the compound of Formula (I), (IA), or (IB), or corresponding pharmaceutically acceptable salts thereof.

Radiotherapy may include one or more of the following categories of therapy:
i. External radiation therapy using electromagnetic radiation (for example focal external beam radiotherapy [“EBRT”]), and intraoperative radiation therapy using electromagnetic radiation;
ii. Internal radiation therapy or brachytherapy; including interstitial radiation therapy or intraluminal radiation therapy; or
iii. Systemic radiation therapy, including but not limited to iodine 131 and strontium 89.

In one embodiment there is provided a compound of Formula (I), or a pharmaceutically acceptable salt thereof, for use in the treatment of cancer, where the compound of Formula (I), or a pharmaceutically acceptable salt thereof is used simultaneously, separately or sequentially with radiotherapy. In one embodiment there is provided a compound of Formula (IA), or a pharmaceutically acceptable salt thereof, for use in the treatment of cancer, where the compound of Formula (IA), or a pharmaceutically acceptable salt thereof is used simultaneously, separately or sequentially with radiotherapy. In one embodiment there is provided a compound of Formula (IA), or a pharmaceutically acceptable salt thereof, for use in the treatment of cancer, where the compound of Formula (IA), or a pharmaceutically acceptable salt thereof is used simultaneously, separately or sequentially with radiotherapy. In any embodiment the cancer is glioblastoma. In any embodiment the radiotherapy is focal external beam radiotherapy.

In one embodiment there is provided a method of treating cancer in a warm-blooded animal who is in need of such treatment, which comprises administering to said warm-blooded animal a compound of Formula (I), or a pharmaceutically acceptable salt thereof and radiotherapy, where the compound of Formula (I), or a pharmaceutically acceptable salt thereof and radiotherapy are jointly effective in producing an anti-cancer effect. In one embodiment there is provided a method of treating cancer in a warm-blooded animal who is in need of such treatment, which comprises administering to said warm-blooded animal a compound of Formula (IA), or a pharmaceutically acceptable salt thereof and radiotherapy, where the compound of Formula (IA), or a pharmaceutically acceptable salt thereof and radiotherapy are jointly effective in producing an anti-cancer effect. In one embodiment there is provided a method of treating cancer in a warm-blooded animal who is in need of such treatment, which comprises administering to said warm-blooded animal a compound of Formula (IB), or a pharmaceutically acceptable salt thereof and radiotherapy, where the compound of Formula (IB), or a pharmaceutically acceptable salt thereof and radiotherapy are jointly effective in producing an anti-cancer effect. In any embodiment the cancer is glioblastoma. In any embodiment the radiotherapy is focal external beam radiotherapy.

In one embodiment there is provided a method of treating cancer in a warm-blooded animal who is in need of such treatment, which comprises administering to said warm-blooded animal a compound of Formula (I), or a pharmaceutically acceptable salt thereof and simultaneously, separately or sequentially administering radiotherapy, where the compound of Formula (I), or a pharmaceutically acceptable salt thereof and radiotherapy are jointly effective in producing an anti-cancer effect. In one embodiment there is provided a method of treating cancer in a warm-blooded animal who is in need of such treatment, which comprises administering to said warm-blooded animal a compound of Formula (IA), or a pharmaceutically acceptable salt thereof and simultaneously, separately or sequentially administering radiotherapy, where the compound of Formula (IA), or a pharmaceutically acceptable salt thereof and radiotherapy are jointly effective in producing an anti-cancer effect. In one embodiment there is provided a method of treating cancer in a warm-blooded animal who is in need of such treatment, which comprises administering to said warm-blooded animal a compound of Formula (IB), or a pharmaceutically acceptable salt thereof and simultaneously, separately or sequentially administering radiotherapy, where the compound of Formula (IB), or a pharmaceutically acceptable salt thereof and radiotherapy are jointly effective in producing an anti-cancer effect. In any embodiment the cancer is glioblastoma.

In any embodiment the radiotherapy is selected from one or more of the categories of radiotherapy listed under points (i)-(iii) above.

Chemotherapy may include one or more of the following categories of anti-tumour substance:
i. Antineoplastic agents and combinations thereof, such as DNA alkylating agents (for example cis-platin, oxaliplatin, carboplatin, cyclophosphamide, nitrogen mustards like ifosfamide, bendamustine, melphalan, chlorambucil, busulphan, temozolamide and nitrosoureas like carmustine); antimetabolites (for example gemcitabine and antifolates such as fluoropyrimidines like 5-fluorouracil and tegafur, raltitrexed, methotrexate, cytosine arabino side, and hydroxyurea); anti-tumour antibiotics (for example anthracyclines like adriamycin, bleomycin, doxorubicin, liposomal doxorubicin, pirarubicin, daunomycin, epirubicin, idarubicin, mitomycin-C, dactinomycin, amrubicin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, irinotecan, topotecan and camptothecin); inhibitors of DNA repair mechanisms such as CHK kinase; DNA-dependent protein kinase inhibitors; inhibitors of poly (ADP-ribose) polymerase (PARP inhibitors, including olaparib); and Hsp90 inhibitors such as tanespimycin and retaspimycin, inhibitors of ATR kinase (such as AZD6738); and inhibitors of WEE1 kinase (such as AZD1775/MK-1775);
ii. Antiangiogenic agents such as those that inhibit the effects of vascular endothelial growth factor, for example the anti-vascular endothelial cell growth factor antibody bevacizumab and for example, a VEGF receptor tyrosine kinase inhibitor such as vandetanib (ZD6474), sorafenib, vatalanib (PTK787), sunitinib (SU11248), axitinib (AG-013736), pazopanib (GW 786034) and cediranib (AZD2171); compounds such as those disclosed in International Patent Applications WO97/22596, WO 97/30035, WO 97/32856 and WO 98/13354; and compounds that work by other mechanisms (for example linomide, inhibitors of integrin αvβ3 function and angiostatin), or inhibitors of angiopoietins and their receptors (Tie-1 and Tie-2), inhibitors of PLGF, inhibitors of delta-like ligand (DLL-4);
iii. Immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor; approaches to decrease T-cell anergy or regulatory T-cell function; approaches that enhance T-cell responses to tumours, such as blocking antibodies to CTLA4 (for example ipilimumab and tremelimumab), B7H1, PD-1 (for example BMS-936558 or AMP-514), PD-L1 (for example MEDI-4736) and agonist antibodies to CD137; approaches using transfected immune cells such as cytokine-transfected dendritic cells; approaches using cytokine-transfected tumour cell lines, approaches using antibodies to tumour associated antigens, and antibodies that deplete target cell types (e.g., unconjugated anti-CD20 antibodies such as Rituximab, radiolabeled anti-CD20 antibodies Bexxar and Zevalin, and anti-CD54 antibody Campath); approaches using anti-idiotypic antibodies; approaches that enhance Natural Killer cell function; and approaches that utilize antibody-toxin conjugates (e.g. anti-CD33 antibody Mylotarg); immunotoxins such as moxetumumab pasudotox; agonists of toll-like receptor 7 or toll-like receptor 9;
iv. Efficacy enhancers, such as leucovorin.

Therefore, in one embodiment there is provided a compound of Formula (I), or a pharmaceutically acceptable salt thereof, for use in the treatment of cancer, where the compound of Formula (I), or a pharmaceutically acceptable salt thereof is used simultaneously, separately or sequentially with at least one additional anti-tumour substance. Therefore, in one embodiment there is provided a compound of Formula (IA), or a pharmaceutically acceptable salt thereof, for use in the treatment of cancer, where the compound of Formula (IA), or a pharmaceutically acceptable salt thereof is used simultaneously, separately or sequentially with at least one additional anti-tumour substance. Therefore, in one embodiment there is provided a compound of Formula (IB), or a pharmaceutically acceptable salt thereof, for use in the treatment of cancer, where the compound of Formula (IB), or a pharmaceutically acceptable salt thereof is used simultaneously, separately or sequentially with at least one additional anti-tumour substance. In any embodiment there is one additional anti-tumour substance. In any embodiment there are two additional anti-tumour substances. In any embodiment there are three or more additional anti-tumour substances.

In one embodiment there is provided a method of treating cancer in a warm-blooded animal who is in need of such treatment, which comprises administering to said warm-blooded animal a compound of Formula (I), or a pharmaceutically acceptable salt thereof and at least one additional anti-tumour substance, where the amounts of the compound of Formula (I), or a pharmaceutically acceptable salt thereof, and the additional anti-tumour substance are jointly effective in producing an anti-cancer effect. In one embodiment there is provided a method of treating cancer in a warm-blooded animal who is in need of such treatment, which comprises administering to said warm-blooded animal a compound of Formula (IA), or a pharmaceutically acceptable salt thereof and at least one additional anti-tumour substance, where the amounts of the compound of Formula (IA), or a pharmaceutically acceptable salt thereof, and the additional anti-tumour substance are jointly effective in producing an anti-cancer effect. In one embodiment there is provided a method of treating cancer in a warm-blooded animal who is in need of such treatment, which comprises administering to said warm-blooded animal a compound of Formula (IB), or a pharmaceutically acceptable salt thereof and at least one additional anti-tumour substance, where the amounts of the compound of Formula (IB), or a pharmaceutically acceptable salt thereof, and the additional anti-tumour substance are jointly effective in producing an anti-cancer effect.

In one embodiment there is provided a method of treating cancer in a warm-blooded animal who is in need of such treatment, which comprises administering to said warm-blooded animal a compound of Formula (I), or a pharmaceutically acceptable salt thereof, and simultaneously, separately or sequentially administering at least one additional anti-tumour substance to said warm-blooded animal, where the amounts of the compound of Formula (I), or pharmaceutically acceptable salt thereof, and the additional anti-tumour substance are jointly effective in producing an anti-cancer effect. In one embodiment there is provided a method of treating cancer in a warm-blooded animal who is in need of such treatment, which comprises administering to said warm-blooded animal a compound of Formula (IA), or a pharmaceutically acceptable salt thereof, and simultaneously, separately or sequentially administering at least one additional anti-tumour substance to said warm-blooded animal, where the amounts of the compound of Formula (IA), or pharmaceutically acceptable salt thereof, and the additional anti-tumour substance are jointly effective in producing an anti-cancer effect. In one embodiment there is provided a method of treating cancer in a warm-blooded animal who is in need of such treatment, which comprises administering to said warm-blooded animal a compound of Formula (IB), or a pharmaceutically acceptable salt thereof, and simultaneously, separately or sequentially administering at least one additional anti-tumour substance to said warm-blooded animal, where the amounts of the compound of Formula (IB), or pharmaceutically acceptable salt thereof, and the additional anti-tumour substance are jointly effective in producing an anti-cancer effect.

In any embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In any embodiment, the cancer is colorectal cancer.

In any embodiment the additional anti-tumour substance is selected from one or more of the anti-tumour substances listed under points (i)-(iv) above.

In one embodiment there is provided a compound of Formula (I), or a pharmaceutically acceptable salt thereof, for use in the treatment of cancer, where the compound of Formula (I), or a pharmaceutically acceptable salt thereof is used simultaneously, separately or sequentially with at least one anti-neoplastic agent. In one embodiment there is provided a compound of Formula (IA), or a pharmaceutically acceptable salt thereof, for use in the treatment of cancer, where the compound of Formula (IA), or a pharmaceutically acceptable salt thereof is used simultaneously, separately or sequentially with at least one anti-neoplastic agent. In one embodiment there is provided a compound of Formula (IB), or a pharmaceutically acceptable salt thereof, for use in the treatment of cancer, where the compound of Formula (IB), or a pharmaceutically acceptable salt thereof is used simultaneously, separately or sequentially with at least one anti-neoplastic agent. In any embodiment the anti-neoplastic agent is selected from the list of antineoplastic agents in point (i) above.

In one embodiment there is provided a compound of Formula (I), or a pharmaceutically acceptable salt thereof, for use in the treatment of cancer, where the compound of Formula (I), or a pharmaceutically acceptable salt thereof, is used simultaneously, separately or sequentially with at least one additional anti-tumour substance selected from cis-platin, oxaliplatin, carboplatin, doxorubicin, pirarubicin, irinotecan, topotecan, amrubicin, epirubicin, etoposide, mitomycin, bendamustine, chlorambucil, cyclophosphamide, ifosfamide, carmustine, melphalan, bleomycin, olaparib, AZD1775 and AZD6738. In one embodiment there is provided a compound of Formula (IA), or a pharmaceutically acceptable salt thereof, for use in the treatment of cancer, where the compound of Formula (IA), or a pharmaceutically acceptable salt thereof, is used simultaneously, separately or sequentially with at least one additional anti-tumour substance selected from cis-platin, oxaliplatin, carboplatin, doxorubicin, pirarubicin, irinotecan, topotecan, amrubicin, epirubicin, etoposide, mitomycin, bendamustine, chlorambucil, cyclophosphamide, ifosfamide, carmustine, melphalan, bleomycin, olaparib, AZD1775 and AZD6738. In one embodiment there is provided a compound of Formula (IB), or a pharmaceutically acceptable salt thereof, for use in the treatment of cancer, where the compound of Formula (IB), or a pharmaceutically acceptable salt thereof, is used simultaneously, separately or sequentially with at least one additional anti-tumour substance selected from cis-platin, oxaliplatin, carboplatin, doxorubicin, pirarubicin, irinotecan, topotecan, amrubicin, epirubicin, etoposide, mitomycin, bendamustine, chlorambucil, cyclophosphamide, ifosfamide, carmustine, melphalan, bleomycin, olaparib, AZD1775 and AZD6738. In any embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In any embodiment, the cancer is colorectal cancer.

In one embodiment there is provided a compound of Formula (I), or a pharmaceutically acceptable salt thereof, for use in the treatment of cancer, where the compound of Formula (I), or a pharmaceutically acceptable salt thereof, is used simultaneously, separately or sequentially with at least one additional anti-tumour substance selected from doxorubicin, irinotecan, topotecan, etoposide, mitomycin, bendamustine, chlorambucil, cyclophosphamide, ifosfamide, carmustine, melphalan and bleomycin. In one embodiment there is provided a compound of Formula (IA), or a pharmaceutically acceptable salt thereof, for use in the treatment of cancer, where the compound of Formula (IA), or a pharmaceutically acceptable salt thereof, is used simultaneously, separately or sequentially with at least one additional anti-tumour substance selected from doxorubicin, irinotecan, topotecan, etoposide, mitomycin, bendamustine, chlorambucil, cyclophosphamide, ifosfamide, carmustine, melphalan and bleomycin. In one embodiment there is provided a compound of Formula (IB), or a pharmaceutically acceptable salt thereof, for use in the treatment of cancer, where the compound of Formula (IB), or a pharmaceutically acceptable salt thereof, is used simultaneously, separately or sequentially with at least one additional anti-tumour substance selected from doxorubicin, irinotecan, topotecan, etoposide, mitomycin, bendamustine, chlorambucil, cyclophosphamide, ifosfamide, carmustine, melphalan and bleomycin. In any embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In any embodiment, the cancer is colorectal cancer.

In one embodiment there is provided a compound of Formula (I), or a pharmaceutically acceptable salt thereof, for use in the treatment of cancer, where the compound of Formula (I), or a pharmaceutically acceptable salt thereof, is used simultaneously, separately or sequentially with irinotecan. In one embodiment there is provided a compound of Formula (IA), or a pharmaceutically acceptable salt thereof, for use in the treatment of cancer, where the compound of Formula (IA), or a pharmaceutically acceptable salt thereof, is used simultaneously, separately or sequentially with irinotecan. In one embodiment there is provided a compound of Formula (IB), or a pharmaceutically acceptable salt thereof, for use in the treatment of cancer, where the compound of Formula (IB), or a pharmaceutically acceptable salt thereof, is used simultaneously, separately or sequentially with irinotecan. In any embodiment the cancer is colorectal cancer. In any embodiment the cancer is gastric cancer.

In one embodiment there is provided a compound of Formula (I), or a pharmaceutically acceptable salt thereof, for use in the treatment of cancer, where the compound of Formula (I), or a pharmaceutically acceptable salt thereof, is used simultaneously, separately or sequentially with FOLFIRI. In one embodiment there is provided a compound of Formula (IA), or a pharmaceutically acceptable salt thereof, for use in the treatment of cancer, where the compound of Formula (IA), or a pharmaceutically acceptable salt thereof, is used simultaneously, separately or sequentially with FOLFIRI. In one embodiment there is provided a compound of Formula (IB), or a pharmaceutically acceptable salt thereof, for use in the treatment of cancer, where the compound of Formula (IB), or a pharmaceutically acceptable salt thereof, is used simultaneously, separately or sequentially with FOLFIRI. In any embodiment the cancer is colorectal cancer. In any embodiment the cancer is gastric cancer.

“FOLFIRI” is a dosage regime involving a combination of leucovorin, 5-fluorouracil and irinotecan.

In one embodiment there is provided a compound of Formula (I), or a pharmaceutically acceptable salt thereof, for use in the treatment of cancer, where the compound of Formula (I), or a pharmaceutically acceptable salt thereof, is used simultaneously, separately or sequentially with a taxoid. In one embodiment there is provided a compound of Formula (IA), or a pharmaceutically acceptable salt thereof, for use in the treatment of cancer, where the compound of Formula (IA), or a pharmaceutically acceptable salt thereof, is used simultaneously, separately or sequentially with a taxoid. In one embodiment there is provided a compound of Formula (IB), or a pharmaceutically acceptable salt thereof, for use in the treatment of cancer, where the compound of Formula (IB), or a pharmaceutically acceptable salt thereof, is used simultaneously, separately or sequentially with a taxoid. In any embodiment the taxoid is paclitaxel or docetaxel. In any embodiment the taxoid is docetaxel. In any embodiment the cancer is gastric cancer.

In one embodiment there is provided a compound of Formula (I), or a pharmaceutically acceptable salt thereof, for use in the treatment of cancer, where the compound of Formula (I), or a pharmaceutically acceptable salt thereof, is used simultaneously, separately or sequentially with topotecan. In one embodiment there is provided a compound of Formula (IA), or a pharmaceutically acceptable salt thereof, for use in the treatment of cancer, where the compound of Formula (IA), or a pharmaceutically acceptable salt thereof, is used simultaneously, separately or sequentially with a taxoid. In one embodiment there is provided a compound of Formula (IB), or a pharmaceutically acceptable salt thereof, for use in the treatment of cancer, where the compound of Formula (IB), or a pharmaceutically acceptable salt thereof, is used simultaneously, separately or sequentially with topotecan. In any embodiment the cancer is lung cancer. In any embodiment the cancer is small cell lung cancer.

In one embodiment there is provided a compound of Formula (I), or a pharmaceutically acceptable salt thereof, for use in the treatment of cancer, where the compound of Formula (I), or a pharmaceutically acceptable salt thereof, is used simultaneously, separately or sequentially with etoposide. In one embodiment there is provided a compound of Formula (IA), or a pharmaceutically acceptable salt thereof, for use in the treatment of cancer, where the compound of Formula (IA), or a pharmaceutically acceptable salt thereof, is used simultaneously, separately or sequentially with etoposide. In one embodiment there is provided a compound of Formula (IB), or a pharmaceutically acceptable salt thereof, for use in the treatment of cancer, where the compound of Formula (IB), or a pharmaceutically acceptable salt thereof, is used simultaneously, separately or sequentially with etoposide. In any embodiment the cancer is lung cancer. In any embodiment the cancer is small cell lung cancer.

In one embodiment there is provided a compound of Formula (I), or a pharmaceutically acceptable salt thereof, for use in the treatment of cancer, where the compound of Formula (I), or a pharmaceutically acceptable salt thereof, is used simultaneously, separately or sequentially with etoposide and a platin. In one embodiment there is provided a compound of Formula (IA), or a pharmaceutically acceptable salt thereof, for use in the treatment of cancer, where the compound of Formula (IA), or a pharmaceutically acceptable salt thereof, is used simultaneously, separately or sequentially with etoposide and a platin. In one embodiment there is provided a compound of Formula (IB), or a pharmaceutically acceptable salt thereof, for use in the treatment of cancer, where the compound of Formula (IB), or a pharmaceutically acceptable salt thereof, is used simultaneously, separately or sequentially with etoposide and a platin. In any embodiment the cancer is small cell lung cancer. In any embodiment the platin is cis-platin, oxaliplatin or carboplatin. In any embodiment the platin is cis-platin. In any embodiment the cancer is lung cancer. In any embodiment the cancer is small cell lung cancer.

In one embodiment there is provided a compound of Formula (I), or a pharmaceutically acceptable salt thereof, for use in the treatment of cancer, where the compound of Formula (I), or a pharmaceutically acceptable salt thereof, is used simultaneously, separately or sequentially with olaparib. In one embodiment there is provided a compound of Formula (IA), or a pharmaceutically acceptable salt thereof, for use in the treatment of cancer, where the compound of Formula (IA), or a pharmaceutically acceptable salt thereof, is used simultaneously, separately or sequentially with olaparib. In one embodiment there is provided a compound of Formula (IB), or a pharmaceutically acceptable salt thereof, for use in the treatment of cancer, where the compound of Formula (IB), or a pharmaceutically acceptable salt thereof, is used simultaneously, separately or sequentially with olaparib. In any embodiment the cancer is gastric cancer.

In one embodiment there is provided a pharmaceutical composition comprising a compound of Formula (I) and at least one additional anti-tumour substance. In one embodiment there is provided a pharmaceutical composition comprising a compound of Formula (IA) and at least one additional anti-tumour substance. In one embodiment there is provided a pharmaceutical composition comprising a compound of Formula (IB) and at least one additional anti-tumour substance. In any embodiment the pharmaceutical composition also comprises at least one pharmaceutically acceptable diluent or carrier. In any embodiment the anti-tumour substance is an anti-neoplastic agent.

In one embodiment there is provided a pharmaceutical composition comprising a compound of Formula (I) and at least one additional anti-tumour substance, for use in the treatment of cancer. In one embodiment there is provided a pharmaceutical composition comprising a compound of Formula (IA) and at least one additional anti-tumour substance, for use in the treatment of cancer. In one embodiment there is provided a pharmaceutical composition comprising a compound of Formula (IB) and at least one additional anti-tumour substance, for use in the treatment of cancer. In any embodiment the pharmaceutical composition also comprises at least one pharmaceutically acceptable diluent or carrier. In any embodiment the anti-tumour substance is an anti-neoplastic agent.

In one embodiment there is provided a kit comprising:

a) A compound of Formula (I), or a pharmaceutically acceptable salt thereof, in a first unit dosage form;

b) A further additional anti-tumour substance in a further unit dosage form;

c) Container means for containing said first and further unit dosage forms; and optionally

d) Instructions for use. In one embodiment there is provided a kit comprising:

a) A compound of Formula (IA), or a pharmaceutically acceptable salt thereof, in a first unit dosage form;

b) A further additional anti-tumour substance in a further unit dosage form;

c) Container means for containing said first and further unit dosage forms; and optionally

d) Instructions for use. In one embodiment there is provided a kit comprising:

a) A compound of Formula (IB), or a pharmaceutically acceptable salt thereof, in a first unit dosage form;

b) A further additional anti-tumour substance in a further unit dosage form;

c) Container means for containing said first and further unit dosage forms; and optionally

d) Instructions for use. In any embodiment the anti-tumour substance comprises an anti-neoplastic agent.

In any embodiment where an anti-neoplastic agent is mentioned, the anti-neoplastic agent is one or more of the agents listed under point (i) above.

The compounds of Formula (I), (IA) or (IB) or corresponding corresponding pharmaceutically acceptable salts thereof, may be used as pharmaceutical compositions, comprising one or more pharmaceutically acceptable diluents or carriers.

Therefore, in one embodiment there is provided a pharmaceutical composition comprising a compound of Formula (I), or a pharmaceutically acceptable salt thereof, and at least one pharmaceutically acceptable diluent or carrier. Therefore, in one embodiment there is provided a pharmaceutical composition comprising a compound of Formula (IA), or a pharmaceutically acceptable salt thereof, and at least one pharmaceutically acceptable diluent or carrier. Therefore, in one embodiment there is provided a pharmaceutical composition comprising a compound of Formula (IB), or a pharmaceutically acceptable salt thereof, and at least one pharmaceutically acceptable diluent or carrier.

The compositions may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing), or as a suppository for rectal dosing. The compositions may be obtained by conventional procedures using conventional pharmaceutical excipients, well known in the art. Thus, compositions intended for oral use may contain, for example, one or more colouring, sweetening, flavouring and/or preservative agents.

In one embodiment there is provided a pharmaceutical composition comprising a compound of Formula (I), or a pharmaceutically acceptable salt thereof, and at least one pharmaceutically acceptable diluent or carrier, for use in therapy. In one embodiment there is provided a pharmaceutical composition comprising a compound of Formula (IA), or a pharmaceutically acceptable salt thereof, and at least one pharmaceutically acceptable diluent or carrier, for use in therapy. In one embodiment there is provided a pharmaceutical composition comprising a compound of Formula (IB), or a pharmaceutically acceptable salt thereof, and at least one pharmaceutically acceptable diluent or carrier, for use in therapy.

In one embodiment there is provided a pharmaceutical composition comprising a compound of Formula (I), or a pharmaceutically acceptable salt thereof, and at least one pharmaceutically acceptable diluent or carrier, for use in the treatment of cancer. In one embodiment there is provided a pharmaceutical composition comprising a compound of Formula (IA), or a pharmaceutically acceptable salt thereof, and at least one pharmaceutically acceptable diluent or carrier, for use in the treatment of cancer. In one embodiment there is provided a pharmaceutical composition comprising a compound of Formula (IB), or a pharmaceutically acceptable salt thereof, and at least one pharmaceutically acceptable diluent or carrier, for use in the treatment of cancer. In any embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In any embodiment, the cancer is colorectal cancer.

In one embodiment there is provided a pharmaceutical composition comprising a compound of Formula (I), or a pharmaceutically acceptable salt thereof, and at least one pharmaceutically acceptable diluent or carrier, for use in the treatment of cancer, where the pharmaceutical composition is used simultaneously, separately or sequentially with at least one additional anti-tumour substance selected from cis-platin, oxaliplatin, carboplatin, doxorubicin, pirarubicin, irinotecan, topotecan, amrubicin, epirubicin, etoposide, mitomycin, bendamustine, chlorambucil, cyclophosphamide, ifosfamide, carmustine, melphalan, bleomycin, olaparib, AZD1775 and AZD6738. In one embodiment there is provided a pharmaceutical composition comprising a compound of Formula (IA), or a pharmaceutically acceptable salt thereof, and at least one pharmaceutically acceptable diluent or carrier, for use in the treatment of cancer, where the pharmaceutical composition is used simultaneously, separately or sequentially with at least one additional anti-tumour substance selected from cis-platin, oxaliplatin, carboplatin, doxorubicin, pirarubicin, irinotecan, topotecan, amrubicin, epirubicin, etoposide, mitomycin, bendamustine, chlorambucil, cyclophosphamide, ifosfamide, carmustine, melphalan, bleomycin, olaparib, AZD1775 and AZD6738. In one embodiment there is provided a pharmaceutical composition comprising a compound of Formula (IB), or a pharmaceutically acceptable salt thereof, and at least one pharmaceutically acceptable diluent or carrier, for use in the treatment of cancer, where the pharmaceutical composition is used simultaneously, separately or sequentially with at least one additional anti-tumour substance selected from cis-platin, oxaliplatin, carboplatin, doxorubicin, pirarubicin, irinotecan, topotecan, amrubicin, epirubicin, etoposide, mitomycin, bendamustine, chlorambucil, cyclophosphamide, ifosfamide, carmustine, melphalan, bleomycin, olaparib, AZD1775 and AZD6738. In any embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In any embodiment, the cancer is colorectal cancer.

In one embodiment there is provided a pharmaceutical composition comprising a compound of Formula (I), or a pharmaceutically acceptable salt thereof, and at least one pharmaceutically acceptable diluent or carrier, for use in the treatment of cancer, where the pharmaceutical composition is used simultaneously, separately or sequentially with at least one additional anti-tumour substance selected from doxorubicin, irinotecan, topotecan, etoposide, mitomycin, bendamustine, chlorambucil, cyclophosphamide, ifosfamide, carmustine, melphalan and bleomycin. In one embodiment there is provided a pharmaceutical composition comprising a compound of Formula (IA), or a pharmaceutically acceptable salt thereof, and at least one pharmaceutically acceptable diluent or carrier, for use in the treatment of cancer, where the pharmaceutical composition is used simultaneously, separately or sequentially with at least one additional anti-tumour substance selected from doxorubicin, irinotecan, topotecan, etoposide, mitomycin, bendamustine, chlorambucil, cyclophosphamide, ifosfamide, carmustine, melphalan and bleomycin. In one embodiment there is provided a pharmaceutical composition comprising a compound of Formula (IB), or a pharmaceutically acceptable salt thereof, and at least one pharmaceutically acceptable diluent or carrier, for use in the treatment of cancer, where the pharmaceutical composition is used simultaneously, separately or sequentially with at least one additional anti-tumour substance selected from doxorubicin, irinotecan, topotecan, etoposide, mitomycin, bendamustine, chlorambucil, cyclophosphamide, ifosfamide, carmustine, melphalan and bleomycin. In any embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In any embodiment, the cancer is colorectal cancer.

In one embodiment there is provided a pharmaceutical composition comprising a compound of Formula (I), or a pharmaceutically acceptable salt thereof, and at least one pharmaceutically acceptable diluent or carrier, for use in the treatment of cancer, where the pharmaceutical composition is used simultaneously, separately or sequentially with irinotecan. In one embodiment there is provided a pharmaceutical composition comprising a compound of Formula (IA), or a pharmaceutically acceptable salt thereof, and at least one pharmaceutically acceptable diluent or carrier, for use in the treatment of cancer, where the pharmaceutical composition is used simultaneously, separately or sequentially with irinotecan. In one embodiment there is provided a pharmaceutical composition comprising a compound of Formula (IA), or a pharmaceutically acceptable salt thereof, and at least one pharmaceutically acceptable diluent or carrier, for use in the treatment of cancer, where the pharmaceutical composition is used simultaneously, separately or sequentially with irinotecan. In one embodiment, the cancer is selected from colorectal cancer, glioblastoma, gastric cancer, ovarian cancer, diffuse large B-cell lymphoma, chronic lymphocytic leukaemia, head and neck squamous cell carcinoma and lung cancer. In one embodiment, the cancer is colorectal cancer.

In any embodiment where cancer is mentioned in a general sense, the cancer may be selected from colorectal cancer, glioblastoma, gastric cancer, ovarian cancer, diffuse large B-cell lymphoma, chronic lymphocytic leukaemia, head and neck squamous cell carcinoma and lung cancer.

In any embodiment where cancer is mentioned in a general sense the following embodiments may apply:

In one embodiment the cancer is colorectal cancer.

In one embodiment the cancer is glioblastoma.

In one embodiment the cancer is gastric cancer.

In one embodiment the cancer is ovarian cancer.

In one embodiment the cancer is diffuse large B-cell lymphoma.

In one embodiment the cancer is chronic lymphocytic leukaemia.

In one embodiment the cancer is head and neck squamous cell carcinoma.

In one embodiment the cancer is lung cancer. In one embodiment the cancer is small cell lung cancer. In one embodiment the cancer is non-small cell lung cancer.

In one embodiment the cancer is metastatic cancer.

In one embodiment the cancer is non-metastatic cancer.

The compound of Formula (I), (IA), (IB) or corresponding pharmaceutically acceptable salts thereof will normally be administered to a warm-blooded animal at a unit dose within the range 0.005-5000 mg/m² body area of the animal, or alternatively approximately 0.001-100 mg/kg, and this normally provides a therapeutically-effective dose. A unit dose form such as a tablet or capsule will usually contain, for example 0.1-250 mg of active ingredient. The daily dose will necessarily be varied depending upon the host treated, the particular route of administration, any therapies being co-administered, and the severity of the illness being treated. Accordingly the practitioner who is treating any particular patient may determine the optimum dosage.

### EXAMPLES

The various embodiments are illustrated by the following Examples. The invention is not to be interpreted as being limited to the Examples. During the preparation of the Examples, generally:
i. Operations were carried out at ambient temperature, i.e. in the range of about 17 to 30° C. and under an atmosphere of an inert gas such as nitrogen unless otherwise stated;
ii. Evaporations were carried out by rotary evaporation or utilising Genevac equipment in vacuo and work-up procedures were carried out after removal of residual solids by filtration;
iii. Flash chromatography purifications were performed on an automated Armen Glider Flash: Spot II Ultimate (Armen Instrument, Saint-Ave, France) or automated Presearch combiflash companions using prepacked Merck normal phase Si60 silica cartridges (granulometry: 15-40 or 40-63 μm) obtained from Merck, Darmstadt, Germany, silicycle silica cartridges or graceresolv silica cartridges;
iv. Preparative chromatography was performed on a Waters instrument (600/2700 or 2525) fitted with a ZMD or ZQ ESCi mass spectrometers and a Waters X-Terra or a Waters X-Bridge or a Waters SunFire reverse-phase column (C-18, 5 microns silica, 19 mm or 50 mm diameter, 100 mm length, flow rate of 40 mL/minute) using decreasingly polar mixtures of water (containing 1% NH₃) and MeCN or decreasingly polar mixtures of water (containing 0.1% formic acid) and MeCN as eluents;
v. Yields, where present, are not necessarily the maximum attainable;
vi. Structures of end-products of Formula (I) were confirmed by nuclear magnetic resonance (NMR) spectroscopy, with NMR chemical shift values measured on the delta scale. Proton magnetic resonance spectra were determined using a Bruker advance 700 (700 MHz), Bruker Avance 500 (500 MHz), Bruker 400 (400 MHz) or Bruker 300 (300 MHz) instrument; 19F NMR were determined at 282 MHz or 376 MHz; 13C NMR were determined at 75 MHz or 100 MHz; measurements were taken at around 20-30° C. unless otherwise specified; the following abbreviations have been used: s=singlet; d=doublet; t=triplet; q=quartet; p=pentet/quintet; m=multiplet; dd=doublet of doublets; ddd=doublet of doublet of doublet; dt=doublet of triplets; td=triplet of doublets; qd=quartet of doublets; bs=broad signal;
vii. End-products of Formula (I) were also characterised by mass spectroscopy following liquid chromatography (LCMS); LCMS was carried out using an Waters Alliance HT (2790 & 2795) fitted with a Waters ZQ ESCi or ZMD ESCi mass spectrometer and an X Bridge 5 μM C-18 column (2.1×50 mm) at a flow rate of 2.4 mL/min, using a solvent system of 95% A+5% C to 95% B+5% C over 4 minutes, where A=water, B=MeOH, C=1:1 MeOH:water (containing 0.2% ammonium carbonate); or by using a Shimadzu UFLC or UHPLC coupled with DAD detector, ELSD detector and 2020 EV mass spectrometer (or equivalent) fitted with a Phenomenex Gemini-NX C18 3.0×50 mm, 3.0 μM column or equivalent (basic conditions) or a Shim pack XR—ODS 3.0×50 mm, 2.2 μM column or Waters BEH C18 2.1×50 mm, 1.7 μM column or equivalent using a solvent system of 95% D+5% E to 95% E+5% D over 4 minutes, where D=water (containing 0.05% TFA), E=MeCN (containing 0.05% TFA) (acidic conditions) or a solvent system of 90% F+10% G to 95% G+5% F over 4 minutes, where F=water (containing 6.5 mm ammonium hydrogen carbonate and adjusted to pH10 by addition of NH₃), G=MeCN (basic conditions);
viii. Intermediates were not generally fully characterised and purity was assessed by thin layer chromatographic, mass spectral, HPLC and/or NMR analysis;
ix. X-ray powder diffraction spectra were determined (using a Panlytical Cubix instrument) by mounting a sample of the crystalline material on a Panalytical single silicon crystal (SSC) wafer mount and spreading out the sample into a thin layer with the aid of a microscope slide. The sample was spun at 30 revolutions per minute (to improve counting statistics) and irradiated with X-rays generated by a copper long-fine focus tube operated at 45 kV and 40 mA with a wavelength of 1.5418 angstroms. The X-ray beam was passed through a 0.04 rad soller slit, then an automatic variable divergence slit set at 20 mm and finally a 20 mm beam mask. The reflected radiation was directed through a 20 mm antiscatter slit and a 0.04 rad soller slit. The sample was exposed for 1.905 seconds per 0.0025067° 2-theta increment (continuous scan mode) over the range 2 degrees to 40 degrees 2-theta in theta-theta mode. The instrument was equipped with an X-Celerator detector. Control and data capture was by means of a Dell Pentium 4HT Workstation operating with X'Pert Industry software;
x. Differential Scanning calorimetry was performed on a TA Instruments Q2000 DSC. Typically, less than 5 mg of material contained in a standard aluminium pan fitted with a lid was heated over the temperature range 25° C. to 300° C. at a constant heating rate of 10° C. per minute. A purge gas using nitrogen was used at a flow rate 50 mL per minute;
xi. The following abbreviations have been used: h=hour(s); r.t.=room temperature (˜17-30° C.); CO₂=carbon dioxide; FCC=flash column chromatography using silica; DCM=dichloromethane; DIPEA=diisopropylethylamine; DMA=N,N-dimethylacetamide; DMF=N,N-dimethylformamide; DMSO=dimethylsulphoxide; eq.=equivalent(s); EtOAc=ethyl acetate; EtOH=ethanol; HATU=1-[bis(dimethylamino)methylene]-1H-1,2,3-triazolo[4,5-b]pyridinium 3-oxide hexafluorophosphate; HCl=hydrogen chloride; IPA=isopropyl alcohol; K₂CO₃=potassium carbonate; MeOH=methanol; MeCN=acetonitrile; MgSO₄=anhydrous magnesium sulphate; NaOH=sodium hydroxide; Na₂SO₄=anhydrous sodium sulphate; NH₃=ammonia; NH₄OH=aqueous ammonia solution; Pd(PPh₃)₄=tetrakis(triphenylphosphine)palladium(0); SCX Strong Cation Exchange; sat.=saturated aqueous solution; THF=tetrahydrofuran; t_{R}=retention time; and
xii. IUPAC names were generated using ‘SmiToSd’, a proprietary program built around the OpenEye Lexichem toolkit (http://www.eyesopen.com/lexichem-tk).

### Example 1

### 6-[6-(3-Dimethylaminopropoxy)pyridin-3-yl]-N-methyl-4-[[(1S)-1-(oxan-4-yl)ethyl]amino]cinnoline-3-carboxamide

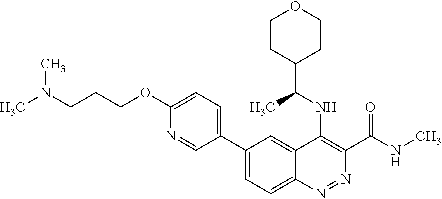

A solution of 3-(dimethylamino)propan-1-ol (0.255 ml, 2.16 mmol) in DMA (9.38 mL) was added to a stirred suspension of sodium hydride (0.194 g, 4.86 mmol) in DMA (9.38 mL) at ambient temperature over a period of 5 minutes under a nitrogen atmosphere. (S)-6-(6-Fluoropyridin-3-yl)-N-methyl-4-((1-(tetrahydro-2H-pyran-4-yl)ethyl)amino)cinnoline-3-carboxamide (0.442 g, 1.08 mmol) was added portionwise and the resulting suspension was stirred for a further 16 h at ambient temperature then at 50° C. for 1 h. The reaction was flask was cooled in an ice-bath and the reaction quenched by the addition of MeOH (10 mL). The MeOH was removed by evaporation and the resultant mixture purified by ion exchange chromatography using an SCX column eluting with 0.35M NH₃/MeOH. The isolated material was further purified by FCC, elution gradient 0 to 10% MeOH in DCM, to afford the desired material as a pale yellow gum which solidified under high vacuum to give a cream solid (0.402 g, 76%). ¹H NMR Spectrum (400 MHz, DMSO-d6): δ 1.28-1.41 (2H, m), 1.36 (3H, d), 1.56 (1H, d), 1.67 (1H, d), 1.76-1.83 (1H, m), 1.87 (2H, tt), 2.14 (6H, s), 2.35 (2H, t), 2.86 (3H, d), 3.21-3.31 (2H, m), 3.80-3.95 (2H, m), 4.20-4.30 (1H, m), 4.35 (2H, t), 6.97 (1H, d), 8.1-8.2 (2H, m), 8.27 (1H, d), 8.32 (1H, s), 8.62 (1H, d), 9.25 (1H, q), 10.25 (1H, d). Mass Spectrum: m/z (ES+)[M+H]+=493.

Experiments were carried out to develop crystalline forms of Example 1. Material obtained as described above was placed in a vial with a magnetic stirrer bar, and approximately 2 mL of IPA added. The vial was then sealed tightly with a cap and left to stir on a magnetic stirrer plate. After approximately 5 days, the sample was removed from the plate, the cap taken off and the slurry left to dry under ambient conditions before it was analysed by XRPD and DSC. This form (Form A) was determined to be crystalline by XRPD, with a melting point of 128.7° C. (onset). Characteristic XRPD peaks for Example 1 Form A are shown in Table 1.

TABLE 1
Characteristic X-Ray powder diffraction peaks for Form A
of 6-[6-(3-Dimethylaminopropoxy)pyridin-3-yl]-N-methyl-
4-[[(1S)-1-(oxan-4-yl)ethyl]amino]cinnoline-3-carboxamide

| Angle 2-Theta (2θ) | Intensity (%) |
|---|---|
| 4.9 | 100 |
| 8.1 | 21 |
| 23.8 | 23 |
| 21.3 | 22 |
| 20.8 | 18 |
| 18.8 | 17 |
| 14.5 | 16 |
| 15.6 | 15 |
| 9.8 | 10 |
| 10.6 | 8 |

In a separate experiment approximately 40 mg of batch 2 material obtained as described above was placed in a vial with a magnetic stirrer bar, and approximately 2 mL of MeCN added. The vial was then sealed tightly with a cap and left to stir on a magnetic stirrer plate. After approximately 5 days, the sample was removed from the plate, the cap taken off and the slurry left to dry under ambient conditions. The resultant solid was then analysed by XRPD and DSC. The solid (“Form B”) was determined to be crystalline by XRPD, with a melting point of 130.0° C. (onset). Characteristic XRPD peaks for Example 1 Form B are shown in Table 2.

TABLE 2
Characteristic X-Ray powder diffraction peaks for Form B
of 6-[6-(3-Dimethylaminopropoxy)pyridin-3-yl]-N-methyl-
4-[[(1S)-1-(oxan-4-yl)ethyl]amino]cinnoline-3-carboxamide

| Angle 2-Theta (2θ) | Intensity (%) |
|---|---|
| 5.4 | 100 |
| 23.4 | 9 |
| 17.6 | 9 |
| 21.6 | 8 |
| 23.2 | 6 |
| 21.9 | 6 |
| 12.6 | 5 |
| 17.0 | 4 |
| 9.5 | 3 |
| 8.9 | 3 |

Example 1 was also prepared on a larger scale as follows. A suspension of sodium hydride (60% dispersion in mineral oil, 10.47 g, 261.81 mmol) and 3-(dimethylamino)propan-1-ol (10.84 mL, 91.63 mmol) in DMA (250 mL) was stirred under nitrogen for 60 minutes. 6-(6-Fluoropyridin-3-yl)-N-methyl-4-[[(1S)-1-(oxan-4-yl)ethyl]amino]cinnoline-3-carboxamide (26.8 g, 65.45 mmol) was added portionwise and additional DMA (20 mL) used to rinse the reactants into the reaction vessel. The reaction mixture was stirred under nitrogen at ambient temperature for 90 minutes then quenched with the addition of saturated ammonium chloride solution (100 mL). The resulting suspension was concentrated under vacuum (65° C., 5 mbar), water (600 mL) added to the residue and the mixture adjusted to pH 10 with the addition of 2M sodium hydroxide solution. The mixture was extracted with DCM (4×500 mL) and the combined organic extracts dried over MgSO₄ and evaporated to dryness. The residue was purified by flash silica chromatography, elution gradient 0 to 6% (10:1 MeOH/conc. NH₃ (aq)) in DCM, to afford the desired material (29.85 g) as a pale yellow foam. This procedure was repeated on a smaller scale, using only 7.0 g (17.10 mmol) of 6-(6-fluoropyridin-3-yl)-N-methyl-4-[[(1S)-1-(oxan-4-yl)ethyl]amino]cinnoline-3-carboxamide and the purified materials combined (37.2 g total). The combined material was subsequently slurried in EtOAc (170 mL) for 3 days and the resulting thick white precipitate collected by filtration, washed with a small amount of cold EtOAc and dried to give the desired material as a white solid (crop 1, 17.1 g). A second crop of desired material was obtained from the liquors as a pale yellow solid (14.3 g). The two crops appeared to have different crystalline forms and hence were recombined along with the residue obtained on evaporating the liquors from the above slurry experiments. This combined material was suspended in EtOAc (75 mL) and the mixture sonicated for 5 minutes. The suspension was stirred at ambient temperature for a further 16 hours and the precipitate collected by filtration and washed with a small amount of cold EtOAc to afford the desired material (28.0 g, 71.5%) as a pale yellow crystalline solid. XRPD analysis determined this material to be Form B. The chiral purity of this material was assessed by a chiral HPLC method in which 1 mg of compound was dissolved in 1 mL of EtOH and analysed by analytical HPLC (Agilent 1100LC system with UV analysis at 280 nM using a Phenomenex Lux C4 column, 5 μm silica, 4.6 mm diameter, 250 mm length), eluting with a 2:1:1 mixture of heptane/EtOH/MeOH with a flow rate of 2 mL/min. The material was found to contain 97.4% of the desired enantiomer and 2.6% of the opposite enantiomer (Example 6; 6-[6-(3-dimethylaminopropoxy)pyridin-3-yl]-N-methyl-4-[[(1R)-1-(oxan-4-yl)ethyl]amino]cinnoline-3-carboxamide).

### Intermediate A: 6-(6-Fluoropyridin-3-yl)-N-methyl-4-[[(1S)-1-(oxan-4-yl)ethyl]amino]cinnoline-3-carboxamide

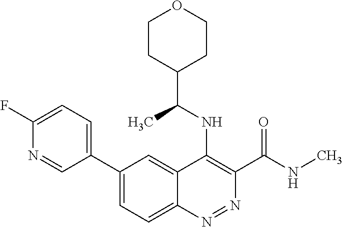 [SMILES: CNC(=O)c1nnc2ccc(-c3ccc(F)nc3)cc2c1N[C@@H](C)C1CCOCC1]

A mixture of 2M potassium carbonate solution (74.4 mL, 148.75 mmol), (6-fluoropyridin-3-yl)boronic acid (9.08 g, 64.46 mmol) and 6-bromo-N-methyl-4-[[(1S)-1-(oxan-4-yl)ethyl]amino]cinnoline-3-carboxamide (Intermediate B, 19.5 g, 49.58 mmol) in isopropyl acetate (550 mL) was purged with nitrogen for 30 minutes. A separate flask was charged with 3-(di-tert-butylphosphino)propane-1-sulfonate (1.331 g, 4.96 mmol) and sodium tetrachloropalladate(II) (0.729 g, 2.48 mmol) in degassed water (60 mL) and stirred at ambient temperature under an inert atmosphere for 30 minutes. The catalyst solution was added to the main reaction mixture and the mixture heated at 90° C. for 18 h under an inert atmosphere before being allowed to cool. The reaction was repeated on an identical scale and the reaction mixture from the two reactions combined. Water (1.2 L) was added and the mixture extracted with EtOAc (3×1.5 L). The organic layers were combined, washed with water (2×1 L), brine (500 mL), dried over Na₂SO₄ and filtered. The mixture was concentrated to approximately 500 mL volume where precipitation was observed. The mixture was heated to 90° C. and further EtOAc added (500 mL) followed by the addition of heptane (˜1 L) and the mixture allowed to cool with stirring. After 16 h stirring, the solid precipitate was collected by filtration and washed with approximately 500 mL of 15% EtOAc in heptane. The solid was dried to afford crude material (˜31 g) as a yellow crystalline solid which was considered may contain palladium residues. The crude material was dissolved in DCM (400 mL) using sonication to aid dissolution. MP-TMT resin (25 g obtained from Biotage AB, Box 8, 75103 Uppsala, Sweden—catalogue number 801471) was added and the mixture was stirred for 20 minutes before being filtered through a plug of silica. The plug/spent resin was eluted with EtOAc and fractions containing the desired material were combined and concentrated to around 500 mL volume. The resulting suspension was stirred for 16 h at ambient temperature then the solid collected by filtration, and washed with a small amount of cold EtOAc to afford the desired material (29.8 g, 73%) as a white crystalline solid. ¹H NMR Spectrum (400 MHz, DMSO-d6): δ 1.29-1.41 (2H, m), 1.36 (3H, d), 1.56 (1H, d), 1.66 (1H, d), 1.74-1.88 (1H, m), 2.87 (3H, d), 3.21-3.31 (2H, m), 3.83-3.93 (2H, m), 4.22-4.33 (1H, m), 7.38 (1H, dd), 8.21 (1H, d), 8.30 (1H, d), 8.38 (1H, s), 8.44 (1H, ddd), 8.71 (1H, s), 9.26 (1H, d), 10.32 (1H, brs). Mass Spectrum: m/z (ES+)[M+H]+=410.

### Intermediate B: 6-Bromo-N-methyl-4-[[(1S)-1-(oxan-4-yl)ethyl]amino]cinnoline-3-carboxamide

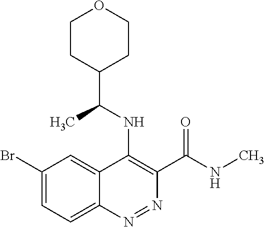 [SMILES: CNC(=O)c1nnc2ccc(Br)cc2c1N[C@@H](C)C1CCOCC1]

DIPEA (36.3 mL, 207.96 mmol) was added to a mixture of 6-bromo-4-chloro-N-methylcinnoline-3-carboxamide (25.0 g, 83.18 mmol), (1S)-1-(oxan-4-yl)ethanamine (Intermediate M, 7.75 g, 60 mmol) and (1S)-1-(oxan-4-yl)ethanamine hydrochloride (5.5 g, 33.20 mmol) in DMA (200 mL) and the resulting mixture stirred at 100° C. for 2 h before being allowed to cool. This procedure was also performed on 17 g (56.57 mmol) of 6-bromo-4-chloro-N-methylcinnoline-3-carboxamide using 8.04 g (62.22 mmol) of (1S)-1-(oxan-4-yl)ethanamine (none of the (1S)-1-(oxan-4-yl)ethanamine hydrochloride was used in this preparation) and the cooled reaction mixture combined with that from the previous preparation. The combined reaction mixtures were partitioned between EtOAc (1.5 L) and water (1.5 L) although the addition of DCM (1 L) was required to ensure all material was in solution. The organic extracts were washed with brine (1.5 L), dried over MgSO₄, filtered and concentrated to around 200 mL volume at which point precipitation was observed. The solid was collected by filtration, washed with a small amount of EtOAc and dried to afford the desired material (40.1 g, 73%) as a white crystalline solid. A second crop of desired material (10.4 g, 19%) was obtained by evaporation of the filtrate and trituration with a small amount of EtOAc. ¹H NMR Spectrum (400 MHz, DMSO-d6): δ1.30 (3H, d), 1.33-1.42 (2H, m), 1.56 (1H, d), 1.62 (1H, dd), 1.73-1.9 (1H, m), 2.87 (3H, d), 3.21-3.27 (2H, m), 3.87-3.91 (2H, m), 4.08-4.12 (1H, m), 7.99 (1H, dd), 8.15 (1H, d), 8.37 (1H, d), 9.24 (1H, d), 10.24 (1H, brs). Mass Spectrum: m/z (ES+)[M+H]+=395.

### Intermediate C: 6-Bromo-4-chloro-N-methylcinnoline-3-carboxamide

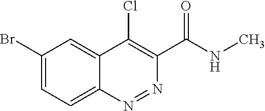 [SMILES: CNC(=O)c1nnc2ccc(Br)cc2c1Cl]

6-Bromo-4-oxo-1H-cinnoline-3-carboxylic acid (34.3 g, 127.48 mmol) was suspended in thionyl chloride (343 mL, 4.7 mol) and DMF (0.983 mL, 12.75 mmol) added. The resulting mixture was stirred at 75° C. for 16 h then the mixture evaporated to dryness and the residue azeotroped three times with toluene. The residue was dissolved in DCM (900 mL) and DIPEA (27.8 mL, 159.36 mmol) and methylamine (2M in THF) (51.0 mL, 101.99 mmol) added dropwise over 30 minutes at 0° C. under an inert atmosphere. The resulting mixture was stirred for a further 15 minutes at 0° C. then additional methylamine (2M in THF) (8.29 mL, 16.57 mmol) added dropwise. The mixture was stirred for a further 15 minutes at 0° C. then diluted with DCM (700 mL) and washed sequentially with water (800 mL), 0.1 M citric acid (800 mL) and saturated sodium hydrogencarbonate solution (400 mL). The organic layer was filtered through a phase-separating paper, EtOAc (1 L) added and the mixture concentrated to 1 L volume. The solid was collected by filtration, washed with a small amount of cold EtOAc and dried to afford the desired material (30.2 g, 79%) as a beige solid. This material was used without further purification but appeared to be contaminated with approximately 10 mol % of 4,6-dichloro-N-methylcinnoline-3-carboxamide. ¹H NMR Spectrum (400 MHz, DMSO-d6): δ 2.91 (3H, d), 8.26 (1H, dd), 8.52 (1H, d), 8.55 (1H, d), 9.00 (1H, q). Mass Spectrum: m/z (ES+)[M+H]+=300.

### Intermediate D: 6-Bromo-4-oxo-1H-cinnoline-3-carboxylic Acid

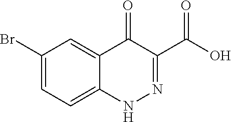 [SMILES: O=C(O)c1n[nH]c2ccc(Br)cc2c1=O]

2M Sodium hydroxide (374 mL, 747.21 mmol) was added to a mixture of ethyl 6-bromo-4-oxo-1H-cinnoline-3-carboxylate (Intermediate E, 44.4 g, 149.44 mmol) in THF (1 L) and MeOH (100 mL) and the resulting mixture stirred at 60° C. for 1 h. Water (600 mL) was added and the mixture heated at 60° C. for a further 2 h. The reaction mixture was diluted with water (1600 mL) and the pH adjusted to pH 3 by the addition of 2M aqueous HCl. The precipitate was collected by filtration, washed with water (1.6 L) and dried under vacuum to afford the desired material (38.6 g, 96%) as a beige solid, which was used without further purification. ¹H NMR Spectrum (400 MHz, DMSO-d6): δ 7.77 (1H, d), 8.10 (1H, dd), 8.31 (1H, d), 14.14 (1H, s), 14.71 (1H, s). Mass Spectrum: m/z (ES+)[M+H]+=267.

### Intermediate E: Ethyl 6-bromo-4-oxo-1H-cinnoline-3-carboxylate

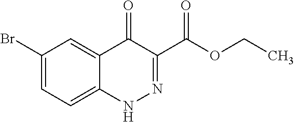 [SMILES: CCOC(=O)c1n[nH]c2ccc(Br)cc2c1=O]

TiCl₄ (116.7 mL, 1.065 mol) was added to a mixture of 2-[(4-bromophenyl)hydrazinylidene]propanedioyl dichloride (Intermediate F, 328.5 g, 1.014 mol) in nitrobenzene (1.64 L) over 10 minutes then the reaction heated to 120° C. for 20 minutes and then cooled to 95° C. and stirred overnight. The reaction was quenched by the dropwise addition of 2M NaOH (4 L, 8 Mol) and the resulting suspension stirred for 2 h. The reaction was filtered twice to remove fine particulates believed to be titanium salts, the filtrate separated and the aqueous layer washed with DCM (3×300 mL). The aqueous layer was filtered again and the filtrate cooled to 5° C. The pH of the mixture was adjusted to pH 1 by the dropwise addition of concentrated aqueous HCl and the resulting slurry stirred for 30 minutes. The mixture was filtered and the solid washed with water (2×100 mL) and dried in a vacuum oven at 40° C. to afford 6-bromo-4-oxo-1H-cinnoline-3-carboxylic acid (102.7 g) containing significant amounts of inorganic impurities and nitrobenzene impurities. Additional impure 6-bromo-4-oxo-1H-cinnoline-3-carboxylic acid (32.2 g) was isolated following a slurry of the titanium salts in 1M NaOH (3 L) for 2 h, at ambient temperature, filtration of the mixture twice to remove fine particulates and the subsequent washing of the aqueous layer with DCM (3×200 mL), filtration, acidification of the filtrate to pH 1 with concentrated aqueous HCl, stirring for 30 minutes and filtration. The impure 6-bromo-4-oxo-1H-cinnoline-3-carboxylic acid (123.17 g) obtained from the above procedure was processed in 3 separate batches according to the following procedure. The impure 6-bromo-4-oxo-1H-cinnoline-3-carboxylic acid was dissolved in EtOH (˜40 volumes) and concentrated sulphuric acid (1.15 equiv) added. The mixture was heated to 90° C. for 5 h then allowed to cool to approximately 50° C. The liquid was removed from insoluble residues by decanting and the residues discarded. The solution was allowed to cool to ambient temperature and stirred for 16 h. The solid was collected by filtration and washed with a small quantity of cold EtOH to afford the desired material (88.08 g over 3 batches) as an orange solid. The filtrates from the 3 procedures were combined and concentrated to approximately 25% of the original volume and cooled to 5° C. to encourage precipitation. The suspension was subsequently stirred at ambient temperature for 16 h, the solid collected by filtration and washed with a small amount of cold EtOH to afford additional desired material (10.7 g). ¹H NMR Spectrum (400 MHz, DMSO-d6): δ 1.29 (3H, t), 4.30 (2H, q), 7.65 (1H, d), 8.00 (1H, dd), 8.18 (1H, d), 14.02 (1H, s). Mass Spectrum: m/z (ES+)[M+H]+=297.

### Intermediate F: 2-[(4-bromophenyl)hydrazinylidene]propanedioyl dichloride

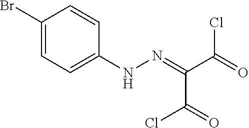 [SMILES: O=C(Cl)C(=NNc1ccc(Br)cc1)C(=O)Cl]

A mixture of 2-[(4-bromophenyl)hydrazinylidene]propanedioic acid (Intermediate G, 170 g, 0.592 mol) and thionyl chloride (510 mL, 7.03 mol) was heated to 40° C. for 44 h and then allowed to cool. The reaction was diluted with heptane (250 mL), filtered and the solid washed with heptane (2×100 mL) to afford the desired material (186.5 g, 97%).

### Intermediate G: 2-[(4-Bromophenyl)hydrazinylidene]propanedioic Acid

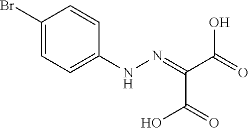 [SMILES: O=C(O)C(=NNc1ccc(Br)cc1)C(=O)O]

A mixture of diethyl 2-[(4-bromophenyl)hydrazinylidene]propanedioate (633 g, 1.84 mol) and EtOH (1265 mL) was heated to 78° C. then 2N NaOH (930 mL, 1.86 mol) added dropwise over 15 minutes maintaining the temperature between 75 and 78° C. Further 1N NaOH (3700 mL, 3.70 mol) was added over 50 minutes maintaining the temperature between 75 and 78° C. the reaction was allowed to cool to ˜55° C. and filtered. The filtrate was allowed to cool to ˜30° C. and was then added dropwise to a solution of concentrated aqueous HCl (563 mL, 6.76 mol) and water (5 L) cooled in a bath of isopropyl alcohol/solid carbon dioxide to maintain the temperature below 10° C. Additional water (1 L) was added and slurry stirred for 30 minutes, filtered and the solid slurried in water (2 L) at ambient temperature for 30 minutes. The suspension was filtered and the solid dried in a vacuum oven at 45° C. for 6 days to afford crude material (494 g). This material was further purified by suspending in EtOAc (2.5 L) and stirring at ambient temperature for 1 h. The mixture was filtered, the solid washed with EtOAc (2×500 mL) and dried in a vacuum oven overnight at 40° C. to afford the desired material (425 g, 81%).

### Intermediate H: Diethyl 2-[(4-bromophenyl)hydrazinylidene]propanedioate

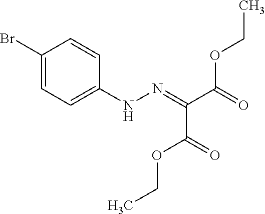 [SMILES: CCOC(=O)C(=NNc1ccc(Br)cc1)C(=O)OCC]

Diethyloxomalonate (349.3 g, 2.01 mol) was added dropwise to a mixture of 4-bromophenyl hydrazine hydrochloride (448.3 g, 2.01 mol) and 50% aqueous EtOH (7800 mL) over 10 minutes and the reaction stirred at ambient temperature overnight. The reaction was diluted with water (4875 mL), stirred for 30 minutes and then filtered. The solid was washed with water (4×500 mL) then dried in a vacuum oven overnight at 40° C. to afford the desired material (633 g) which was used without further purification.

Ethyl 6-bromo-4-oxo-1H-cinnoline-3-carboxylate (Intermediate E) can also be prepared in the manner described below:

A mixture of potassium carbonate (5.44 g, 39.36 mmol) and ethyl (2Z)-3-(5-bromo-2-fluorophenyl)-2-hydrazinylidene-3-oxopropanoatee (Intermediate I, 6.24 g, 19.68 mmol) in DMA (60 mL) was stirred at 100° C. for 3 h. The reaction mixture was allowed to cool, diluted with water (100 mL) and the mixture adjusted to a neutral pH by the addition of 2M aqueous HCl. The precipitate was collected by filtration, washed with water (50 mL) and dried under vacuum to afford the desired material (4.05 g, 69%) as a solid, which was used without further purification. Analytical data was consistent with material prepared by the route previously described.

### Intermediate I: Ethyl (2Z)-3-(5-bromo-2-fluorophenyl)-2-hydrazinylidene-3-oxopropanoate

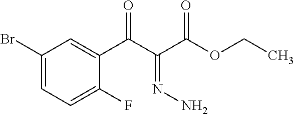 [SMILES: CCOC(=O)/C(=N\N)C(=O)c1cc(Br)ccc1F]

Trimethylphosphine (2.206 mL, 21.40 mmol) was added to a solution of 1-(5-bromo-2-fluorophenyl)-3-ethoxy-1,3-dioxopropane-2-diazonium (Intermediate J, 6.13 g, 19.45 mmol) in THF (55 mL) at ambient temperature under an inert atmosphere and the reaction stirred for 2 h. The reaction mixture was quenched with water (60 mL), extracted with EtOAc (3×70 mL), the organic layer dried over MgSO₄, filtered and evaporated to afford the desired material (6.24 g, 101%) as a mixture of cis and trans isomers. ¹H NMR Spectrum (400 MHz, DMSO-d6): δ 1.05 (1H, t), 1.25 (2H, t), 4.25 (2H, q), 7.17-7.29 (1H, m), 7.59 (1H, dd), 7.61-7.69 (1H, m), 10.54 (2H, s). Mass Spectrum: m/z (ES+)[M+H]+=317.

### Intermediate J: 1-(5-Bromo-2-fluorophenyl)-3-ethoxy-1,3-dioxopropane-2-diazonium

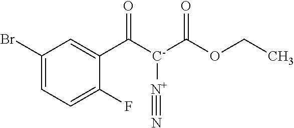 [SMILES: CCOC(=O)[C-]([N+]#N)C(=O)c1cc(Br)ccc1F]

4-Acetamidobenzenesulfonyl azide (4.57 g, 19.02 mmol) was added portionwise to ethyl 3-(5-bromo-2-fluorophenyl)-3-oxopropanoate (5.00 g, 17.30 mmol) and triethylamine (4.34 mL, 31.13 mmol) in MeCN (70 mL) at ambient temperature and the mixture stirred for 18 h. The mixture was filtered, the solid discarded and the filtrate concentrated. The residue was dissolved in EtOAc (250 mL) and washed sequentially with a saturated aqueous solution of ammonium chloride (100 mL) and brine (50 mL). The organic layer was dried over MgSO₄, filtered and concentrated to afford the desired material (6.13 g, 112%) which contained traces of N-(4-sulfamoylphenyl)acetamide and unreacted starting material but was used without further purification. ¹H NMR Spectrum (400 MHz, DMSO-d6): δ 1.14 (3H, t), 4.15 (2H, q), 7.25-7.39 (1H, m), 7.67 (1H, dd), 7.76 (1H, m).

Ethyl 6-bromo-4-oxo-1H-cinnoline-3-carboxylate (Intermediate E) can also be prepared in the manner described below:

TFA (837 mL, 10.863 mol) was added slowly to ethyl 3-(5-bromo-2-pyrrolidin-1-yldiazenylphenyl)-3-oxopropanoate (Intermediate K, 160 g, 434.52 mmol) over a period of 30 minutes at 0° C. under an inert atmosphere. The resulting solution was stirred at ambient temperature for 16 h then the reaction mixture poured onto ice water (2 L). The precipitate was collected by filtration, washed with water (5×100 mL) and dried in the vacuum oven to afford the desired material (118 g, 91%) as a pale yellow solid, which was used without further purification. Analytical data was consistent with material prepared by the routes previously described.

### Intermediate K: Ethyl 3-(5-bromo-2-pyrrolidin-1-yldiazenylphenyl)-3-oxopropanoate

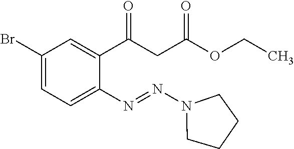 [SMILES: CCOC(=O)CC(=O)c1cc(Br)ccc1/N=N/N1CCCC1]

Sodium hydride (55.3 g, 1382.68 mmol) was added portionwise to a solution of diethyl carbonate (467 g, 3.951 mol) in THF (800 mL) at ambient temperature under an inert atmosphere. A solution of 1-(5-bromo-2-pyrrolidin-1-yldiazenylphenyl)ethanone (Intermediate L|, 117 g, 395.05 mmol) in THF (200 mL) was added slowly over a period of 60 minutes under an inert atmosphere and the resulting mixture stirred at 75° C. for 3 h. The reaction mixture was allowed to cool then quenched with water (100 mL) and the resulting mixture concentrated under vacuum. The residue was diluted with water (500 mL), extracted with EtOAc (4×500 mL), the organic layer dried over Na₂SO₄, filtered and evaporated to afford the desired material (168 g, 115%) as a brown solid, which was used without further purification. ¹H NMR Spectrum (400 MHz, DMSO-d6): δ 1.11 (3H, t), 1.93-2.04 (4H, m), 3.60 (2H, t), 3.93 (2H, t), 4.03 (2H, q), 4.11 (2H, s), 7.41 (1H, d), 7.61-7.64 (2H, m). Mass Spectrum: m/z (ES+)[M+H]+=368.1.

### Intermediate L: 1-(5-Bromo-2-pyrrolidin-1-yldiazenylphenyl)ethanone

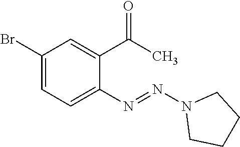 [SMILES: CC(=O)c1cc(Br)ccc1/N=N/N1CCCC1]

1-(2-Amino-5-bromophenyl)ethanone (94.8 g, 442.87 mmol) was added to 2M aqueous HCl (700 mL, 1.40 mol), and the resulting mixture was stirred at 60° C. for 2 h. The mixture was cooled to 0° C. and a solution of sodium nitrite (30.6 g, 442.87 mmol) in water (100 mL) was added dropwise. After 15 minutes the mixture was filtered, the solid discarded and the filtrate added to a stirred solution of pyrrolidine (31.5 g, 442.87 mmol) and sodium hydroxide (56.0 g, 1399.46 mmol) in water (500 mL) at 0° C. After 15 minutes the precipitate was collected by filtration, washed with water and dried in the vacuum oven to afford the desired material (117 g, 89%) as a red solid, which was used without further purification. ¹H NMR Spectrum (400 MHz, DMSO-d6): δ 1.99 (4H, m), 2.54 (3H, s), 3.58 (2H, t), 3.91 (2H, t), 7.37-7.66 (3H, m). Mass Spectrum: m/z (ES+)[M+H]+=298.

(1S)-1-(Oxan-4-yl)ethanamine and (1S)-1-(oxan-4-yl)ethanamine hydrochloride are compounds known in the literature and their preparation has been described (e.g. Antonios-McCrea, W. R. et al., WO2012101062). In addition (1S)-1-(oxan-4-yl)ethanamine is commercially available, for instance from Fluorochem Ltd, Unit 14, Graphite Way, Hadfield, Derbyshire, SK13 1QH, UK (catalogue number 301787). In addition to the procedure described in the literature (1S)-1-(oxan-4-yl)ethanamine can also be prepared in the following manner.

### Intermediate M: (1S)-1-(Oxan-4-yl)ethanamine

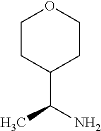

(R)-2-Methyl-N-[(1S)-1-(oxan-4-yl)ethyl]propane-2-sulfinamide (Intermediate N, 381 g, believed to be the borane adduct) was added portionwise over 5 minutes to a 4 M solution of hydrogen chloride in MeOH (2.5 L, 10 mol) at 10° C. and the resulting mixture was stirred at approximately 10° C. for 2.25 h. The bulk of the MeOH was removed under reduced pressure to give a two phase mixture. The oil was dissolved in water (750 mL) and washed with DCM (3×300 mL). The aqueous phase was pH adjusted to 7 by addition of sodium hydrogen carbonate (120 g) and washed with DCM (2×200 mL). The aqueous phase was pH adjusted to ˜13 by addition of sodium hydroxide (60 g), extracted with DCM (3×300 mL) and the combined extracts dried (Na₂SO₄), filtered and concentrated under reduced pressure to afford the desired material (69 g) as a yellow oil. In order to improve the enantiomeric purity of the sample the isolated product (69 g) was dissolved in EtOH (690 mL) and water (288 mL) and L-aspartic acid (71.1 g, 534.2 mmol) added under an inert atmosphere. The mixture was heated to reflux for 30 minutes and the hot mixture filtered. The filtrate was allowed to cool and stand overnight then diluted with EtOH (1.5 L) and the suspension filtered and the solid washed with EtOH (500 mL). The solids were dried under reduced pressure (55° C.) to give a white solid (105 g) which was dissolved a mixture of water (200 mL), brine (100 mL) and sodium hydroxide solution (50% w/v, 100 mL). The solution was extracted with DCM (3×100 ml), the combined extracts dried (Na₂SO₄), filtered and concentrated under reduced pressure (40° C.) to afford the desired material (46.5 g) as a pale yellow liquid. ¹H NMR Spectrum (300 MHz, CDCl₃): δ 0.95 (3H, d), 1.15 (2H, brs), 1.25 (3H, m), 1.4-1.65 (2H, m), 2.63 (1H, m), 3.31 (2H, m), 3.97 (2H, m).

### (R)-2-methyl-N-[(1S)-1-(oxan-4-yl)ethyl]propane-2-sulfinamide (Intermediate N)

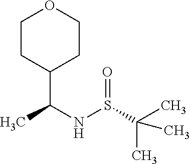

L-Selectride (1 M in THF, 500 mL, 500 mmol, 1.54 eq.) was added over 30 minutes to a mixture of 2-methyl-N-[1-(oxan-4-yl)ethylidene]propane-2-sulfinamide (Intermediate O, 75 g, 324.0 mmol) in THF (940 mL) at −78° C. under an inert atmosphere. After 2 h the mixture was warmed to ambient temperature and stirred for 10 minutes. The mixture was cooled to 10° C. then water (20 mL) in THF (80 mL) was added slowly maintaining the temperature below 15° C. This procedure was repeated on an identical scale and the reaction mixtures combined and the bulk of the solvent removed under reduced pressure (40-50° C.). The resulting cloudy oil was dissolved in DCM (1.2 L) and was washed with water (2×300 mL). The organic layer was dried (Na₂SO₄), filtered and concentrated to give a cloudy oil which was further filtered to afford the desired material (295 g) as a pale yellow oil. This material is believed to contain borane species which may flammable. The material was used without further purification.

### (R)-2-Methyl-N-[1-(oxan-4-yl)ethylidene]propane-2-sulfinamide (Intermediate O)

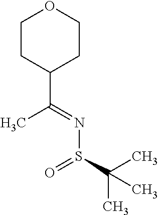

(R)-2-Methylpropanesulfinamide (106.9 g, 882.0 mmol) and titanium tetraethoxide (201.6 g, 883.6 mmol) were added to a solution of 4-acetyltetrahydropyran (112.5 g, 877.7 mmol) in THF (1.4 L) under an inert atmosphere and the mixture heated to reflux for 18 h. The mixture was allowed to cool and poured in to brine (850 mL). The resulting slurry was diluted with EtOAc (1 L) and the mixture filtered through celite. The resulting two phases were separated. The filter cake was washed with EtOAc (4×1 L) and the combined organics dried (Na₂SO₄), filtered and concentrated under vacuum (40-45° C.) to give a cloudy oil that was filtered to afford the desired material (192.5 g, 95%) as a yellow oil which was used without further purification.

6-[6-(3-Dimethylaminopropoxy)pyridin-3-yl]-N-methyl-4-[[(1S)-1-(oxan-4-yl)ethyl]amino]cinnoline-3-carboxamide (AZ13732641) (Example 1) can also be prepared directly from 6-bromo-N-methyl-4-[[(1S)-1-(oxan-4-yl)ethyl]amino]cinnoline-3-carboxamide (Intermediate B) according to the procedure described below.

Pd(PPh₃)₄ (1.175 g, 1.02 mmol) was added to a mixture of 6-bromo-N-methyl-4-[[(1S)-1-(oxan-4-yl)ethyl]amino]cinnoline-3-carboxamide (4 g, 10.17 mmol), N,N-dimethyl-3-[5-(4,4,5,5-tetramethyl-1,3,2-dioxaborolan-2-yl)pyridin-2-yl]oxypropan-1-amine (Intermediate P, 4.05 g, 13.22 mmol) and cesium carbonate (6.63 g, 20.34 mmol) in 1,4-dioxane (20 mL) and water (4 mL) under an inert atmosphere. The resulting mixture was stirred at 90° C. for 3 h then allowed to cool. The reaction mixture was poured onto water (50 mL), extracted with DCM (3×75 mL) and the organic layer evaporated. The crude material was purified by flash C18-flash chromatography, elution gradient 3 to 20% MeCN in water, to afford the desired material (2.2 g, 40%) as a yellow solid. Analytical data was consistent with material prepared by the route previously described.

### Intermediate P: N,N-Dimethyl-3-[5-(4,4,5,5-tetramethyl-1,3,2-dioxaborolan-2-yl)pyridin-2-yl]oxypropan-1-amine

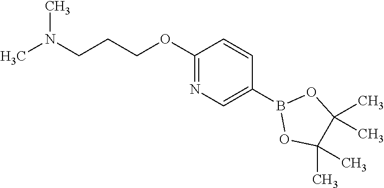

n-Butyllithium (2.5N, 4.8 mL, 50.96 mmol) was added to a solution of 3-(5-bromopyridin-2-yl)oxy-N,N-dimethylpropan-1-amine (Intermediate Q, 2.07 g, 7.99 mmol) and 4,4,5,5-tetramethyl-2-(propan-2-yloxy)-1,3,2-dioxaborolane (2.79 g, 15.00 mmol) in THF (20 mL) at −78° C. over 10 minutes under an inert atmosphere. The resulting solution was stirred for 4 h at 18° C. The reaction was then quenched by the addition of a saturated aqueous solution of ammonium chloride then partitioned between EtOAc (100 mL) and water (100 mL). The organic layer was concentrated in vacuo and the residue purified by FCC, eluting with EtOAc/petroleum ether (1:3) to afford the desired material (270 mg, 11%) as a yellow solid. Mass Spectrum: m/z (ES+)[M+H]+=225.

### Intermediate Q: 3-(5-Bromopyridin-2-yl)oxy-N,N-dimethylpropan-1-amine

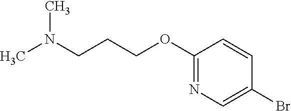

3-(Dimethylamino)propan-1-ol (3.09 g, 29.95 mmol) was added to a mixture of sodium hydride (2.4 g, 60.00 mmol) in DMF (50 mL) over a period of 20 min at ambient temperature. 5-Bromo-2-fluoropyridine (5.81 g, 33.01 mmol) was added and the resulting solution stirred for 4 h at 30° C. The reaction was then quenched by the addition of a saturated aqueous solution of ammonium chloride and the resulting mixture concentrated under vacuum. The residue was purified by FCC, eluting with DCM/MeOH ether (10:1) to afford the desired material (5.2 g, 67%) as yellow oil. Mass Spectrum: m/z (ES+)[M+H]+=259.

### Example 2

### 6-[6-(3-Dimethylaminopropoxy)pyridin-3-yl]-4-[[(1S)-1-(oxan-4-yl)ethyl]amino]cinnoline-3-carboxamide

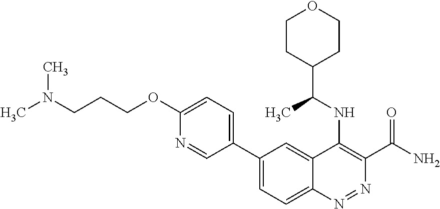 [SMILES: C[C@H](Nc1c(C(N)=O)nnc2ccc(-c3ccc(OCCCN(C)C)nc3)cc12)C1CCOCC1]

A solution of 3-(dimethylamino)propan-1-ol (1.315 mL, 11.12 mmol) in DMA (10 mL) was added dropwise to a stirred suspension of sodium hydride (1.27 g, 31.76 mmol) in DMA (40 mL) at ambient temperature and the resulting suspension stirred for 20 minutes under an inert atmosphere. 6-(6-Fluoropyridin-3-yl)-4-[[(1S)-1-(oxan-4-yl)ethyl]amino]cinnoline-3-carboxamide (Intermediate R, 3.14 g, 7.94 mmol) was added and the reaction stirred at ambient temperature for 1 h and then heated to 50° C. for 10 minutes. The reaction mixture was diluted with DCM (150 mL), and washed sequentially with water (2×100 mL) and saturated brine (100 mL). The organic layer was dried over MgSO₄, filtered and evaporated to afford crude product which was purified by ion exchange chromatography, using an SCX column eluting with 1M NH₃ in MeOH. The material was further purified further by FCC, elution gradient 0 to 20% MeOH in DCM, to afford the desired material (2.1 g, 55%). ¹H NMR Spectrum (400 MHz, CDCl₃): δ 1.34-1.57 (5H, m), 1.62-1.93 (3H, m), 1.93-2.08 (2H, m), 2.28 (6H, s), 2.41-2.53 (2H, m), 3.36-3.40 (2H, m), 3.97-4.03 (2H, m), 4.08-4.20 (1H, m), 4.43 (2H, t), 5.57 (1H, d), 6.89 (1H, d), 7.84 (1H, dd), 7.95 (1H, dd), 8.21 (1H, d), 8.36-8.40 (2H, m), 8.44 (1H, d), 10.20 (1H, d). Mass Spectrum: m/z (ES+)[M+H]+=479.

### Intermediate R: 6-(6-Fluoropyridin-3-yl)-4-[[(1S)-1-(oxan-4-yl)ethyl]amino]cinnoline-3-carboxamide

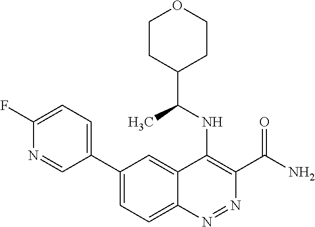 [SMILES: C[C@H](Nc1c(C(N)=O)nnc2ccc(-c3ccc(F)nc3)cc12)C1CCOCC1]

A 1:2 mixture of sodium tetrachloropalladate and 3-(di-tert-butylphosphino)propane-1-sulfonic acid (0.05 M in water) (9.91 mL, 0.50 mmol) was added to 6-bromo-4-[[(1S)-1-(oxan-4-yl)ethyl]amino]cinnoline-3-carboxamide (Intermediate S, 3.76 g, 9.91 mmol), (6-fluoropyridin-3-yl)boronic acid (1.537 g, 10.91 mmol) and potassium carbonate (4.11 g, 29.74 mmol) in degassed 1,4-dioxane (70 mL) and water (17.5 mL) under an inert atmosphere. The resulting mixture was stirred at 80° C. for 18 h then allowed to cool. The reaction mixture was diluted with EtOAc (200 mL), and washed sequentially with water (2×200 mL) and saturated brine (100 mL). The organic layer was dried over MgSO₄, filtered and evaporated to afford the desired material (3.39 g, 86%) as a pale yellow solid. ¹H NMR Spectrum (400 MHz, DMSO-d6): δ 1.34-1.38 (5H, m), 1.54 (1H, d), 1.65 (1H, d), 1.76-1.83 (1H, m), 3.25 (2H, t), 3.8-3.95 (2H, m), 4.19-4.33 (1H, m), 7.38 (1H, dd), 7.74 (1H, s), 8.21 (1H, d), 8.30 (1H, d), 8.36 (1H, s), 8.44 (1H, td), 8.61 (1H, s), 8.71 (1H, d), 10.34 (1H, s). Mass Spectrum: m/z (ES+)[M+H]+=396.

### Intermediate S: 6-Bromo-4-[[(1S)-1-(oxan-4-yl)ethyl]amino]cinnoline-3-carboxamide

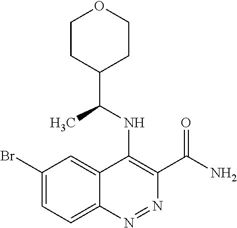 [SMILES: C[C@H](Nc1c(C(N)=O)nnc2ccc(Br)cc12)C1CCOCC1]

DIPEA (4.47 mL, 25.57 mmol) was added in one portion to 6-bromo-4-chlorocinnoline-3-carboxamide (2.93 g, 10.23 mmol) and (1S)-1-(oxan-4-yl)ethanamine hydrochloride (1.864 g, 11.25 mmol) in DMA (40 mL). The resulting mixture was stirred at 100° C. for 2 h. The reaction mixture was diluted with EtOAc (500 mL), and washed sequentially with water (2×200 mL) and saturated brine (100 mL). The organic layer was dried over MgSO₄, filtered and evaporated to afford the desired material (3.76 g, 97%). ¹H NMR Spectrum (400 MHz, DMSO-d6): δ 1.32 (5H, d), 1.59 (2H, dd), 1.76 (1H, s), 3.25 (2H, t), 3.75-3.96 (2H, m), 3.99-4.14 (1H, m), 7.76 (1H, s), 7.99 (1H, dd), 8.14 (1H, d), 8.34 (1H, s), 8.61 (1H, s), 10.26 (1H, s). Mass Spectrum: m/z (ES+)[M+H]+=396.

### Intermediate T: 6-Bromo-4-chlorocinnoline-3-carboxamide

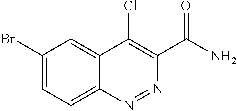 [SMILES: NC(=O)c1nnc2ccc(Br)cc2c1Cl]

Ammonium hydroxide (35.5 mL, 910.43 mmol) was added dropwise over a period of 10 minutes to a solution of 6-bromo-4-chloro-N-methylcinnoline-3-carboxamide (Intermediate C, 3.80 g, 12.42 mmol) in acetone (60 mL) at 0° C. The resulting mixture was stirred at ambient temperature for 30 minutes then the precipitate collected by filtration, washed with acetone (10 mL) and dried under vacuum to afford the desired material (2.93 g, 82%) as a solid, which was used without further purification. ¹H NMR Spectrum (400 MHz, DMSO-d6): δ 8.11 (1H, s), 8.26 (1H, dd), 8.41 (1H, s), 8.49-8.68 (2H, m Mass Spectrum: m/z (ES+)[M+H]+=286.

The preparation of (1S)-1-(oxan-4-yl)ethanamine hydrochloride and 6-bromo-4-chloro-N-methylcinnoline-3-carboxamide have been described earlier.

6-[6-(3-Dimethylaminopropoxy)pyridin-3-yl]-4-[[(1S)-1-(oxan-4-yl)ethyl]amino]cinnoline-3-carboxamide (AZ13713471) (Example 2) can also be prepared directly from 6-bromo-4-[[(1S)-1-(oxan-4-yl)ethyl]amino]cinnoline-3-carboxamide (Intermediate S) according to the procedure described below.

Pd(PPh₃)₄ (1,219 g, 1.05 mmol) was added to a mixture of 6-bromo-4-[[(1S)-1-(oxan-4-yl)ethyl]amino]cinnoline-3-carboxamide (4 g, 10.55 mmol), N,N-dimethyl-3-[5-(4,4,5,5-tetramethyl-1,3,2-dioxaborolan-2-yl)pyridin-2-yl]oxypropan-1-amine (Intermediate P, 4.20 g, 13.71 mmol) and cesium carbonate (6.87 g, 21.09 mmol) in 1,4-dioxane (10 mL) and water (2 mL) under an inert atmosphere. The resulting mixture was stirred at 90° C. for 3 h then allowed to cool. The reaction mixture was poured onto water (50 mL), extracted with DCM (3×75 mL) and the organic layer evaporated. The crude material was purified by flash C18-flash chromatography, elution gradient 3 to 20% MeCN in water, to afford the desired material (3.5 g, 63%) as a yellow solid. Analytical data was consistent with material prepared by the route previously described.

The preparation of N,N-dimethyl-3-[5-(4,4,5,5-tetramethyl-1,3,2-dioxaborolan-2-yl)pyridin-2-yl]oxypropan-1-amine (Intermediate P) was described previously.

### Example 3

### 4-[[(1S)-1-(Oxan-4-yl)ethyl]amino]-6-[6-(3-pyrrolidin-1-ylpropoxy)pyridin-3-yl]cinnoline-3-carboxamide

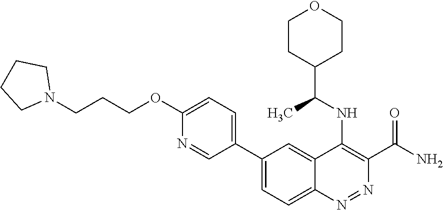 [SMILES: C[C@H](Nc1c(C(N)=O)nnc2ccc(-c3ccc(OCCCN4CCCC4)nc3)cc12)C1CCOCC1]

A solution of 3-(pyrrolidin-1-yl)propan-1-ol (174 mg, 1.35 mmol) in DMA (6 mL) was added dropwise to a stirred suspension of sodium hydride (154 mg, 3.84 mmol) in DMA (6 mL) at ambient temperature under an inert atmosphere and the resulting suspension stirred for 20 minutes. 6-(6-Fluoropyridin-3-yl)-4-[[(1S)-1-(oxan-4-yl)ethyl]amino]cinnoline-3-carboxamide (380 mg, 0.96 mmol) was added and the reaction stirred at ambient temperature for 18 h. Water (5 mL) was added and the crude material purified by ion exchange chromatography, using an SCX column eluting with 1M NH₃ in MeOH. The isolated material was further purified by FCC, elution gradient 0 to 15% MeOH in DCM, to afford the desired material (353 mg, 72.8%) as a yellow foam. ¹H NMR Spectrum (400 MHz, DMSO-d6): δ 1.32-1.41 (5H, m), 1.56 (1H, d), 1.61-1.75 (5H, m), 1.76-1.82 (1H, m), 1.93 (2H, p), 2.49-2.62 (6H, m), 3.22-3.33 (2H, m), 3.79-3.97 (2H, m), 4.17-4.33 (1H, m), 4.39 (2H, t), 6.98 (1H, dd), 7.71 (1H, s), 8.13-8.18 (2H, m), 8.24-8.36 (2H, m), 8.59 (1H, s), 8.64 (1H, d), 10.27 (1H, d). Mass Spectrum: m/z (ES+)[M+H]+=505.

The preparation of 6-(6-fluoropyridin-3-yl)-4-[[(1S)-1-(oxan-4-yl)ethyl]amino]cinnoline-3-carboxamide (Intermediate R) has been described previously.

4-[[(1S)-1-(Oxan-4-yl)ethyl]amino]-6-[6-(3-pyrrolidin-1-ylpropoxy)pyridin-3-yl]cinnoline-3-carboxamide (AZ13733400) (Example 3) can also be prepared directly from 6-bromo-4-[[(1S)-1-(oxan-4-yl)ethyl]amino]cinnoline-3-carboxamide (Intermediate S) according to the procedure described below.

Pd(PPh₃)₄ (54.8 mg, 0.05 mmol) was added to a mixture of 6-bromo-4-[[(1S)-1-(oxan-4-yl)ethyl]amino]cinnoline-3-carboxamide (180 mg, 0.47 mmol), 2-(3-pyrrolidin-1-ylpropoxy)-5-(4,4,5,5-tetramethyl-1,3,2-dioxaborolan-2-yl)pyridine (Intermediate U, 315 mg, 0.95 mmol) and cesium carbonate (309 mg, 0.95 mmol) in 1,4-dioxane (5 mL) and water (1 mL) under an inert atmosphere. The resulting mixture was stirred at 90° C. for 2 h then allowed to cool. The reaction mixture was poured onto water (15 mL), extracted with EtOAc (3×15 mL) and the organic layer washed with brine then evaporated. The crude material was purified by preparative HPLC (XBridge Prep C18 OBD column, 5 μm silica, 19 mm diameter, 150 mm length), using decreasingly polar mixtures of water (containing 0.1% NH₃) and MeCN as eluents, to afford the desired material (85 mg, 36%) as a white solid. Analytical data was consistent with material prepared by the route previously described.

### Intermediate U: 2-(3-Pyrrolidin-1-ylpropoxy)-5-(4,4,5,5-tetramethyl-1,3,2-dioxaborolan-2-yl)pyridine

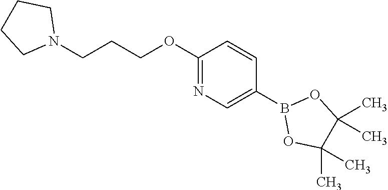 [SMILES: CC1(C)OB(c2ccc(OCCCN3CCCC3)nc2)OC1(C)C]

n-Butyllithium (5.68 mL, 14.20 mmol) was added dropwise to a mixture of 5-bromo-2-(3-pyrrolidin-1-ylpropoxy)pyridine (Intermediate V, 2.7 g, 9.47 mmol) and 2-isopropoxy-4,4,5,5-tetramethyl-1,3,2-dioxaborolane (2.64 g, 14.20 mmol) in THF (20 mL) at −78° C. over a period of 10 minutes under an inert atmosphere. The resulting mixture was allowed to warm to ambient temperature and stirred for 12 h. The reaction mixture was quenched by the addition of a saturated aqueous solution of ammonium chloride, extracted with EtOAc (2×50 mL) and the organic layer dried over Na₂SO₄, filtered and evaporated to afford the desired material (3.10 g, 99%) as a yellow oil. The product was used in the next step directly without further purification. ¹H NMR Spectrum (400 MHz, CDCl₃): δ 1.26-1.41 (12H, m), 1.77-1.80 (4H, m), 1.95-2.04 (2H, m), 2.50-2.58 (4H, m), 2.62 (2H, t), 4.37 (2H, t), 6.69 (1H, d), 7.91 (1H, d), 8.52 (1H, s). Mass Spectrum: m/z (ES+)[M+H]+=251.

### Intermediate V: 5-bromo-2-(3-pyrrolidin-1-ylpropoxy)pyridine

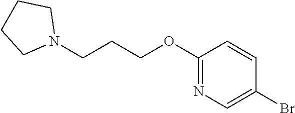 [SMILES: Brc1ccc(OCCCN2CCCC2)nc1]

Sodium hydride (0.591 g, 14.77 mmol) was added portionwise to a solution of 3-(pyrrolidin-1-yl)propan-1-ol (1.615 g, 12.50 mmol) in THF (20 mL) at to 0° C. then stirred at ambient temperature for 30 minutes. 5-Bromo-2-fluoropyridine (2 g, 11.36 mmol) was added and the resulting mixture stirred at ambient temperature for 2 h before being quenched by the addition of a saturated aqueous solution of ammonium chloride. The mixture was extracted with EtOAc (2×100 mL), the organic layer dried over Na₂SO₄, filtered and evaporated to afford pale yellow solid. The crude product was purified by FCC, elution gradient 0 to 10% MeOH in DCM, to afford the desired material (2.70 g, 83%) as a yellow solid. Mass Spectrum: m/z (ES+)[M+H]+=285.

### Example 4

### 6-[6-(3-Methylaminopropoxy)pyridin-3-yl]-4-[[(1S)-1-(oxan-4-yl)ethyl]amino]cinnoline-3-carboxamide

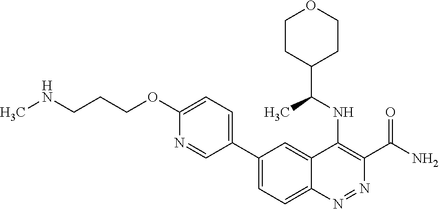 [SMILES: CNCCCOc1ccc(-c2ccc3nnc(C(N)=O)c(N[C@@H](C)C4CCOCC4)c3c2)cn1]

4.0 M Hydrogen chloride in dioxane (1.868 mL, 7.47 mmol) was added to tert-butyl N-[3-[5-[3-carbamoyl-4-[[(1S)-1-(oxan-4-yl)ethyl]amino]cinnolin-6-yl]pyridin-2-yl]oxypropyl]-N-methylcarbamate (Intermediate W, 422 mg, 0.75 mmol) and the mixture stirred at ambient temperature for 1 h. The reaction mixture was evaporated to dryness and the residue purified by ion exchange chromatography, using an SCX column eluting with 1 M NH₃ in MeOH. The isolated material was further purified by FCC, elution gradient 0 to 10% (1 M NH₃ in MeOH) in DCM, to afford the desired material (140 mg, 40%) as a cream foam. ¹H NMR Spectrum (400 MHz, DMSO-d6): δ 1.35-1.39 (5H, m), 1.56 (1H, d), 1.67 (1H, d), 1.73-1.84 (1H, m), 1.86-1.92 (2H, m), 2.31 (3H, s), 2.64 (2H, t), 3.13-3.5 (3H, m), 3.76-4 (2H, m), 4.15-4.31 (1H, m), 4.39 (2H, t), 6.98 (1H, d), 7.71 (1H, s), 8.15-8.19 (2H, m), 8.25-8.38 (2H, m), 8.51-8.68 (2H, m), 10.27 (1H, d). Mass Spectrum: m/z (ES+)[M+H]+=465.

### Intermediate W: tert-Butyl N-[3-[5-[3-carbamoyl-4-[[(1S)-1-(oxan-4-yl)ethyl]amino]cinnolin-6-yl]pyridin-2-yl]oxypropyl]-N-methylcarbamate

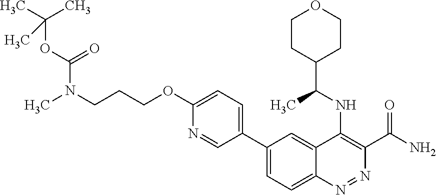 [SMILES: C[C@H](Nc1c(C(N)=O)nnc2ccc(-c3ccc(OCCCN(C)C(=O)OC(C)(C)C)nc3)cc12)C1CCOCC1]

A solution of tert-butyl N-(3-hydroxypropyl)-N-methylcarbamate (168 mg, 0.89 mmol) in DMA (2.0 mL) was added dropwise to a stirred suspension of sodium hydride (101 mg, 2.53 mmol) in DMA (4 mL) under an inert atmosphere and the resulting mixture stirred for 20 minutes. 6-(6-Fluoropyridin-3-yl)-4-[[(1S)-1-(oxan-4-yl)ethyl]amino]cinnoline-3-carboxamide (Intermediate R, 250 mg, 0.63 mmol) was added and the reaction stirred at ambient temperature for 1 h and then heated to 50° C. for 1 h. Water (5 mL) was added and the crude product purified by ion exchange chromatography, using an SCX column eluting with 1 M NH₃ in MeOH. The isolated material was further purified by FCC, elution gradient 0 to 5% MeOH in DCM, to afford the desired material (422 mg, 118%) as a gummy solid which was used without further purification. ¹H NMR Spectrum (400 MHz, DMSO-d6): δ 1.21-1.46 (14H, m), 1.51-1.62 (1H, m), 1.67 (1H, d), 1.76-1.83 (1H, m), 1.94-1.98 (2H, m), 2.27-2.37 (1H, m), 2.54-2.6 (1H, m), 2.78-2.82 (2H, m), 3.35 (1H, t), 3.46 (1H, t), 3.85-3.89 (2H, m), 4.03-4.07 (1H, m), 4.17-4.3 (1H, m), 4.34 (2H, t), 6.98 (1H, d), 7.71 (1H, s), 8.11-8.25 (2H, m), 8.25-8.37 (2H, m), 8.55-8.69 (2H, m), 10.27 (1H, d). Mass Spectrum: m/z (ES+)[M+H]+=565.

The preparation of 6-(6-fluoropyridin-3-yl)-4-[[(1S)-1-(oxan-4-yl)ethyl]amino]cinnoline-3-carboxamide (Intermediate R) was described earlier.

### Example 5

### N-Methyl-6-[6-(3-methylaminopropoxy)pyridin-3-yl]-4-[[(1S)-1-(oxan-4-yl)ethyl]amino]cinnoline-3-carboxamide

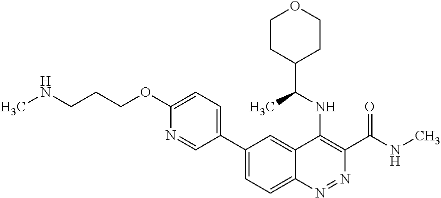

4.0 M Hydrogen chloride in dioxane (0.121 mL, 0.48 mmol) was added to tert-Butyl N-methyl-N-[3-[5-[3-(methylcarbamoyl)-4-[[(1S)-1-(oxan-4-yl)ethyl]amino]cinnolin-6-yl]pyridin-2-yl]oxypropyl]carbamate (Intermediate X, 280 mg, 0.48 mmol) and the mixture stirred at ambient temperature for 1 h. The reaction mixture was evaporated to dryness and the residue purified by ion exchange chromatography, using an SCX column eluting with 1 M NH₃ in MeOH. The isolated material was further purified by FCC, elution gradient 0 to 10% (1 M NH₃ in MeOH) in DCM, to afford the desired material (109 mg, 47%) as a cream foam. ¹H NMR Spectrum (400 MHz, DMSO-d6): δ 1.36 (5H, d), 1.56 (1H, d), 1.67 (1H, d), 1.79-1.84 (1H, m), 1.87 (2H, p), 2.29 (3H, s), 2.61 (2H, t), 2.86 (3H, d), 3.21-3.38 (3H, m), 3.84-3.90 (2H, m), 4.11-4.32 (1H, m), 4.37 (2H, t), 6.97 (1H, d), 8.12-8.19 (2H, m), 8.27 (1H, d), 8.32 (1H, s), 8.62 (1H, d), 9.25 (1H, d), 10.24 (1H, d). Mass Spectrum: m/z (ES+)[M+H]+=479.

### Intermediate X: tert-Butyl N-methyl-N-[3-[5-[3-(methylcarbamoyl)-4-[[(1S)-1-(oxan-4-yl)ethyl]amino]cinnolin-6-yl]pyridin-2-yl]oxypropyl]carbamate

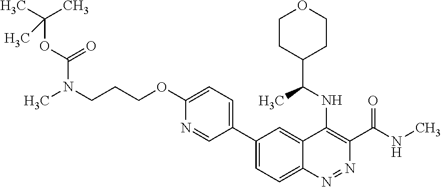 [SMILES: CNC(=O)c1nnc2ccc(-c3ccc(OCCCN(C)C(=O)OC(C)(C)C)nc3)cc2c1N[C@@H](C)C1CCOCC1]

A solution of tert-butyl N-(3-hydroxypropyl)-N-methylcarbamate (129 mg, 0.68 mmol) in DMA (4.0 mL) was added dropwise to a stirred suspension of sodium hydride (78 mg, 1.95 mmol) in DMA (4 mL) under an inert atmosphere and the resulting mixture stirred for 20 minutes. 6-(6-Fluoropyridin-3-yl)-N-methyl-4-[[(1S)-1-(oxan-4-yl)ethyl]amino]cinnoline-3-carboxamide (Intermediate A, 200 mg, 0.49 mmol) was added and the reaction stirred at ambient temperature for 15 minutes and then heated to 50° C. for 1 h. Water (5 mL) was added and the crude product purified by ion exchange chromatography, using an SCX column eluting with 1 M NH₃ in MeOH. The isolated material was further purified by FCC, elution gradient 0 to 5% MeOH in DCM, to afford the desired material (280 mg, 99%) as a gummy solid. ¹H NMR Spectrum (400 MHz, DMSO-d6): δ 1.33-1.40 (15H, m), 1.58 (1H, d), 1.68 (1H, d), 1.74-1.9 (1H, m), 2.81 (2H, s), 2.88 (3H, d), 3.23-3.38 (6H, m), 3.88 (2H, t), 4.19-4.3 (1H, m), 4.34 (2H, t), 6.97 (1H, d), 8.17 (2H, dd), 8.28 (1H, d), 8.33 (1H, d), 8.63 (1H, d), 9.24 (1H, q), 10.25 (1H, d). Mass Spectrum: m/z (ES+)[M+H]+=579.

The preparation of 6-(6-fluoropyridin-3-yl)-N-methyl-4-[[(1S)-1-(oxan-4-yl)ethyl]amino]cinnoline-3-carboxamide (Intermediate A) was described previously.

### Example 6

6-[6-(3-Dimethylaminopropoxy)pyridin-3-yl]-N-methyl-4-[[(1R)-1-(oxan-4-yl)ethyl]amino]cinnoline-3-carboxamide

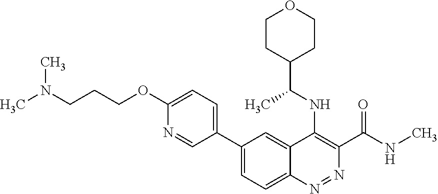

A solution of 3-(dimethylamino)propan-1-ol (35.3 mg, 0.34 mmol) in DMA (1.5 mL) was added dropwise to sodium hydride (60% dispersion in oil, 39.1 mg, 0.98 mmol) suspended in DMA (3 mL) under an inert atmosphere and the resulting mixture stirred at ambient temperature for 20 minutes. 6-(6-Fluoropyridin-3-yl)-N-methyl-4-[[(1R)-1-(oxan-4-yl)ethyl]amino]cinnoline-3-carboxamide (Intermediate Y, 100 mg, 0.24 mmol) was added and the reaction mixture stirred at ambient temperature for 3 h before being heated to 50° C. for 8 h. The reaction mixture was poured into water (50 mL) and the pH adjusted to pH 9 with 2M aqueous HCl. The mixture was extracted with EtOAc (3×50 mL) and the combined organic extracts washed with brine (30 mL), dried over MgSO₄ and evaporated. The residue was purified by FCC, elution gradient 3 to 5% (10:1 MeOH/conc. NH3 (aqueous)) in DCM, to afford the desired material (28 mg, 23%) as a white solid. ¹H NMR Spectrum (400 MHz, DMSO-d6): δ 1.36 (3H, d), 1.41 (2H, s), 1.56 (1H, d), 1.67 (1H, d), 1.75-1.83 (1H, m), 1.88 (2H, tt), 2.15 (6H, s), 2.36 (2H, t), 2.86 (3H, d), 3.22-3.30 (2H, m), 3.81-3.93 (2H, m), 4.25 (1H, d), 4.35 (2H, t), 6.97 (1H, d), 8.12-8.20 (2H, m), 8.27 (1H, d), 8.32 (1H, s), 8.62 (1H, d), 9.25 (1H, q), 10.26 (1H, d). Mass Spectrum: m/z (ES+)[M+H]+=493.

### Intermediate Y: 6-(6-Fluoropyridin-3-yl)-N-methyl-4-[[(1R)-1-(oxan-4-yl)ethyl]amino]cinnoline-3-carboxamide

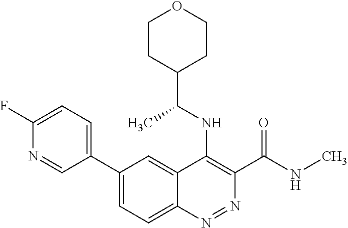 [SMILES: CNC(=O)c1nnc2ccc(-c3ccc(F)nc3)cc2c1N[C@H](C)C1CCOCC1]

A 1:2 mixture of sodium tetrachloropalladate and 3-(di-tert-butylphosphino)propane-1-sulfonic acid (0.05 M in water) (0.509 mL, 0.03 mmol) was added to 6-bromo-4-[[(1R)-1-(oxan-4-yl)ethyl]amino]cinnoline-3-carboxamide (Intermediate Z, 200 mg, 0.51 mmol), (6-fluoropyridin-3-yl)boronic acid (86 mg, 0.61 mmol) and 2 M potassium carbonate solution (0.763 mL, 1.53 mmol) in 1,4-dioxane (5 mL) and water (1.25 mL). The resulting mixture was stirred at 80° C. for 12 h in a microwave reactor then allowed to cool. The reaction mixture was partitioned between water (50 mL) and EtOAc (50 mL), and the organic layer washed with water (50 mL) and saturated brine (25 mL). The organic layer was dried over MgSO₄, filtered and evaporated and the residue purified by FCC, eluting with 40-80% EtOAc in heptane, to afford the desired material (180 mg, 86%) as a solid. ¹H NMR Spectrum (400 MHz, DMSO-d6): δ 1.36 (3H, d), 1.3-1.43 (2H, m), 1.56 (1H, d), 1.66 (1H, d), 1.74-1.87 (1H, m), 2.87 (3H, d), 3.21-3.30 (2H, m), 3.82-3.92 (2H, m), 4.22-4.32 (1H, m), 7.38 (1H, dd), 8.20 (1H, d), 8.30 (1H, d), 8.38 (1H, s), 8.44 (1H, ddd), 8.71 (1H, d), 9.26 (1H, q), 10.31 (1H, s). Mass Spectrum: m/z (ES+)[M+H]+=410.

### Intermediate Z: 6-Bromo-N-methyl-4-[[(1R)-1-(oxan-4-yl)ethyl]amino]cinnoline-3-carboxamide

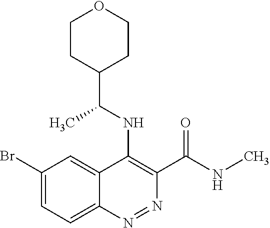 [SMILES: CNC(=O)c1nnc2ccc(Br)cc2c1N[C@H](C)C1CCOCC1]

DIPEA (0.157 mL, 0.90 mmol) was added to a mixture of 6-bromo-4-chloro-N-methylcinnoline-3-carboxamide (200 mg, 0.60 mmol) and (1R)-1-(oxan-4-yl)ethanamine (0.090 mL, 0.66 mmol) in DMA (5 mL) and the resulting mixture stirred at 100° C. for 2 h. The reaction mixture was diluted with EtOAc (500 mL), and washed sequentially with water (2×200 mL) and brine (100 mL). The organic layer was dried over MgSO₄, filtered and evaporated. The residue was purified by FCC, elution gradient 30 to 70% EtOAc in heptane, to afford the desired material (215 mg, 91%) as a solid which was used without further purification. ¹H NMR Spectrum (400 MHz, DMSO-d6): δ 1.30 (3H, d), 1.34-1.40 (2H, m), 1.56 (1H, d), 1.65 (1H, d), 1.71-1.84 (1H, m), 2.85 (3H, d), 3.20-3.30 (2H, m), 3.82-3.93 (2H, m), 4.05-4.15 (1H, m), 7.98 (1H, d), 8.13 (1H, d), 8.36 (1H, s), 9.25 (1H, q), 10.24 (1H, s). Mass Spectrum: m/z (ES+)[M+H]+=393.

The preparation of 6-bromo-4-chloro-N-methylcinnoline-3-carboxamide (Intermediate C) has been described earlier.

(1R)-1-(Oxan-4-yl)ethanamine and (1R)-1-(oxan-4-yl)ethanamine hydrochloride are compounds known in the literature and their preparation has been described (e.g. Antonios-McCrea, W. R. et al., WO2012101062). In addition (1R)-1-(oxan-4-yl)ethanamine is commercially available, for instance from Fluorochem Ltd, Unit 14, Graphite Way, Hadfield, Derbyshire, SK13 1QH, UK (catalogue number 301768).

### Biological Assays

An ATM cellular potency assay was used to measure the effects of the compounds of the present invention. During the description of the assay, generally:
i. The following abbreviations have been used: Ab=Antibody; BSA=Bovine Serum Albumin; CO₂=Carbon Dioxide; DMEM=Dulbecco's Modified Eagle Medium; DMSO=Dimethyl Sulphoxide; EMEM=Eagle's Minimal Essential Medium; FBS=Foetal Bovine Serum; h=hour(s); PBS=Phosphate buffered saline.
ii. IC₅₀ values were calculated using a smart fitting model in Genedata. The IC₅₀ value was the concentration of test compound that inhibited 50% of biological activity.

### Assay a): ATM Cellular Potency

### Rationale:

Cellular irradiation induces DNA double strand breaks and rapid intermolecular autophosphorylation of serine 1981 that causes dimer dissociation and initiates cellular ATM kinase activity. Most ATM molecules in the cell are rapidly phosphorylated on this site after doses of radiation as low as 0.5 Gy, and binding of a phosphospecific antibody is detectable after the introduction of only a few DNA double-strand breaks in the cell.

The rationale of the pATM assay is to identify inhibitors of ATM in cells. HT29 cells are incubated with test compounds for 1 hr prior to X-ray-irradiation. 1 h later the cells are fixed and stained for pATM (Ser1981). The fluorescence is read on the arrayscan imaging platform.

### Method Details:

HT29 cells (ECACC #85061109) were seeded into 384 well assay plates (Costar #3712) at a density of 3500 cells/well in 40 μl EMEM medium containing 1% L glutamine and 10% FBS and allowed to adhere overnight. The following morning compounds of Formula (I) in 100% DMSO were added to assay plates by acoustic dispensing. After 1 h incubation at 37° C. and 5% CO₂, plates (up to 6 at a time) were irradiated using the X-RAD 320 instrument (PXi) with equivalent to ˜600 cGy. Plates were returned to the incubator for a further 1 h. Then cells were fixed by adding 20 μl of 3.7% formaldehyde in PBS solution and incubating for 20 minutes at r.t. before being washed with 50 μl/well PBS, using a Biotek EL405 plate washer. Then 20 μl of 0.1% Triton X100 in PBS was added and incubated for 20 minutes at r.t., to permeabalise cells. Then the plates were washed once with 50 μl/well PBS, using a Biotek EL405 plate washer.

Phospho-ATM Ser1981 antibody (Millipore #MAB3806) was diluted 10000 fold in PBS containing 0.05% polysorbate/Tween and 3% BSA and 20 μl was added to each well and incubated over night at r.t. The next morning plates were washed three times with 50 μl/well PBS, using a Biotek EL405 plate washer, and then 20 μl of secondary Ab solution, containing 500 fold diluted Alexa Fluor® 488 Goat anti-rabbit IgG (Life Technologies, A11001) and 0.002 mg/ml Hoeschst dye (Life technologies #H-3570), in PBS containing 0.05% polysorbate/Tween and 3% BSA, was added. After 1 h incubation at r.t., the plates were washed three times with 50 μl/well PBS, using a Biotek EL405 plate washer, and plates were sealed and kept in PBS at 4° C. until read. Plates were read using an ArrayScan VTI instrument, using an XF53 filter with 10× objective. A two laser set up was used to analyse nuclear staining with Hoeschst (405 nm) and secondary antibody staining of pSer1981 (488 nm).

The results of testing the Examples in assay a) are shown in Table 3.

TABLE 3
Potency Data for Examples 1-6 in Assay a)

| Example | Assay a) ATM Cell IC₅₀ (μM) |
|---|---|
| 1 | 0.00274 |
| 2 | 0.00187 |
| 3 | 0.00178 |
| 4 | 0.00772 |
| 5 | 0.00491 |
| 6 | 0.0089 |

1. A compound of Formula (I): 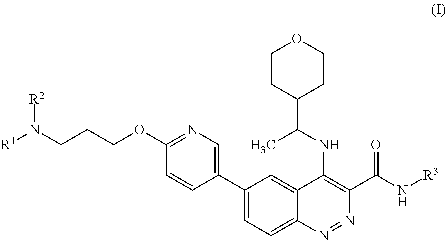
Or a pharmaceutically acceptable salt thereof, where:
R¹ is (C₁-C₃)alkyl;
R² is hydro or (C₁-C₃)alkyl; or
R¹ and R² together with the nitrogen atom to which they are attached form an azetidinyl, pyrrolidinyl, or piperidinyl ring; and
R³ is hydro or methyl.

2. The compound of Formula (I), or a pharmaceutically acceptable salt thereof, as claimed in claim 1, where R¹ is (C₁-C₃)alkyl and R² is hydro or (C₁-C₃)alkyl; or R¹ and R² together with the nitrogen atom to which they are attached form a pyrrolidinyl ring.

3. The compound of Formula (I), or a pharmaceutically acceptable salt thereof, as claimed in claim 1, where R¹ is methyl.

4. The compound of Formula (I), or a pharmaceutically acceptable salt thereof, as claimed in claim 1, where R² is hydro or methyl.

5. The compound of Formula (I), or a pharmaceutically acceptable salt thereof, as claimed in claim 4, where R² is methyl.

6. The compound of Formula (I), or a pharmaceutically acceptable salt thereof, as claimed in any one of claims 1 to 5, where R³ is hydro.

7. The compound of Formula (I), or a pharmaceutically acceptable salt thereof, as claimed in any one of claims 1 to 5, where R³ is methyl.

8. The compound of Formula (I), or a pharmaceutically acceptable salt thereof, as claimed in claim 1, where the compound is selected from:
6-[6-(3-Dimethylaminopropoxy)pyridin-3-yl]-N-methyl-4-[[(1S)-1-(oxan-4-yl)ethyl]amino]cinnoline-3-carboxamide;
6-[6-(3-Dimethylaminopropoxy)pyridin-3-yl]-4-[[(1S)-1-(oxan-4-yl)ethyl]amino]cinnoline-3-carboxamide;
4-[[(1S)-1-(Oxan-4-yl)ethyl]amino]-6-[6-(3-pyrrolidin-1-ylpropoxy)pyridin-3-yl]cinnoline-3-carboxamide;
6-[6-(3-Methylaminopropoxy)pyridin-3-yl]-4-[[(1S)-1-(oxan-4-yl)ethyl]amino]cinnoline-3-carboxamide;
N-Methyl-6-[6-(3-methylaminopropoxy)pyridin-3-yl]-4-[[(1S)-1-(oxan-4-yl)ethyl]amino]cinnoline-3-carboxamide; and
6-[6-(3-Dimethylaminopropoxy)pyridin-3-yl]-N-methyl-4-[[(1R)-1-(oxan-4-yl)ethyl]amino]cinnoline-3-carboxamide.

9. A pharmaceutical composition which comprises a compound of Formula (I), or a pharmaceutically acceptable salt thereof, as claimed in any one of claims 1 to 8, and at least one pharmaceutically acceptable diluent or carrier.

10. A compound of Formula (I), or a pharmaceutically acceptable salt thereof, as claimed in any one of claims 1 to 8, for use in therapy.

11. A compound of Formula (I), or a pharmaceutically acceptable salt thereof, as claimed in any one of claims 1 to 8, for use in the treatment of cancer.

12. The use according to claim 11, where the compound of Formula (I) is used simultaneously, separately or sequentially with radiotherapy.

13. The use according to claim 11, where the compound of Formula (I) is used simultaneously, separately or sequentially with at least one additional anti-tumour substance selected from doxorubicin, irinotecan, topotecan, etoposide, mitomycin, bendamustine, chlorambucil, cyclophosphamide, ifosfamide, carmustine, melphalan and bleomycin.

14. Use of a compound of Formula (I), or a pharmaceutically acceptable salt thereof, as claimed in any one of claims 1 to 8, for the manufacture of a medicament for the treatment of cancer.

15. A method for treating cancer in a warm-blooded animal in need of such treatment, which comprises administering to said warm-blooded animal a therapeutically effective amount of a compound of Formula (I), or a pharmaceutically acceptable salt thereof, as claimed in any one of claims 1 to 8.
